(19) United States
(12) Patent Application Publication
(10) Pub. No.: US 2023/0363851 A1
(43) Pub. Date: Nov. 16, 2023

(54) METHODS AND SYSTEMS FOR VIDEO COLLABORATION

(71) Applicant: Avail Medsystems, Inc., Santa Clara, CA (US)

(72) Inventors: Daniel HAWKINS, Palo Alto, CA (US); Ravi KALLURI, San Jose, CA (US); Arun KRISHNA, Pleasanton, CA (US); Shivakumar MAHADEVAPPA, Fremont, CA (US)

(21) Appl. No.: 18/046,703

(22) Filed: Oct. 14, 2022

Related U.S. Application Data
(63) Continuation of application No. PCT/US21/28101, filed on Apr. 20, 2021.
(60) Provisional application No. 63/012,394, filed on Apr. 20, 2020.
(60) Provisional application No. 63/121,701, filed on Dec. 4, 2020.

(51) Int. Cl. A61B 90/00; A61B 34/10; A61B 34/20; G16H 30/20

(52) U.S. Cl. CPC A61B 90/361; A61B 34/10; A61B 34/20; G16H 30/20; A61B 2034/2055

(57) ABSTRACT

The present disclosure provides methods and systems for video collaboration. The method may comprise (a) obtaining a plurality of videos of a surgical procedure; (b) determining an amount of progress for the surgical procedure based at least in part on the plurality of videos; and (c) updating an estimated timing of one or more steps of the surgical procedure based at least in part on the amount of progress. The method may further comprise providing the estimating timing to one or more end users to coordinate another surgical procedure or patient room turnover. In some cases, the method may comprise (a) obtaining a plurality of videos of a surgical procedure and (b) providing the plurality of videos to a plurality of end users, wherein each end user of the plurality of end users receives a different subset of the plurality of videos.

### CROSS-REFERENCE

This application is a continuation of International Patent Application PCT/US21/28101, filed on Apr. 20, 2021, which claims priority to U.S. Provisional Application No. 63/012,394, filed on Apr. 20, 2020, and U.S. Provisional Application No. 63/121,701, filed on Dec. 4, 2020, each of which is incorporated herein by reference in its entirety for all purposes.

### BACKGROUND

Medical practitioners may perform various procedures within a medical suite, such as an operating room. Often times, there may be minimal communication with other individuals who are not physically present in the operating room. Even if medical practitioners do wish to provide updates on an ongoing medical procedure to individuals outside the operating room, there may be limited resources and options for doing so. This may hinder coordination and/or communications between medical practitioners in the operating room and other medical practitioners who are outside the operating room. Further, medical practitioners in an operating room may be unable to quickly provide timely and accurate updates on the medical procedure to other individuals outside the operating room.

### SUMMARY

A need exists for improved systems and methods of video collaboration to enhance communication and coordination between individuals in an operating room and individuals outside an operating room. A need exists for systems and methods that allow for medical practitioners, friends, families, vendors, or other medical personnel (e.g., support staff or hospital administrators) to effectively and quickly track, monitor, and evaluate a performance or completion of one or more steps of a medical operation or surgical procedure using videos obtained and/or streamed during the medical operation or surgical procedure. Recognized herein are various limitations with systems and methods currently available for video collaboration, such as in the context of medical operations and surgical procedures. The systems and methods of the present disclosure may enable medical practitioners in an operating room to selectively provide timely and accurate updates on the medical procedure to other individuals located remotely from the operating room. The systems and methods of the present disclosure may enable medical practitioners in an operating room to provide video data associated with one or more steps of a medical operation to one or more end users located outside of the operating room. The systems and methods of the present disclosure may also enable the sharing of different kinds of video data with different end users based on the relevancy of such video data to each end user. The systems and methods of the present disclosure may further enable the sharing of different kinds of video data to help coordinate parallel procedures (e.g., concurrent donor and recipient surgical procedures) or to help coordinate patient room turnover in a medical facility such as a hospital. In some cases, the systems and methods of the present disclosure may be used to broadcast video data to end users for educational or training purposes. In some cases, the systems and methods of the present disclosure may be used to generate educational or informative content based on a plurality of videos obtained using one or more imaging devices. In some cases, the systems and methods of the present disclosure may be used to distribute such educational or informative content to medical practitioners, doctors, physicians, nurses, surgeons, medical operators, medical personnel, medical staff, medical students, medical interns, and/or medical residents to aid in medical education or medical practice.

In an aspect, the present disclosure provides methods for video collaboration. The method may comprise (a) obtaining a plurality of videos of a surgical procedure; (b) determining an amount of progress for the surgical procedure based at least in part on the plurality of videos; and (c) updating an estimated timing of one or more steps of the surgical procedure based at least in part on the amount of progress. In some embodiments, the method may further comprise providing the estimating timing to one or more end users to coordinate another surgical procedure. In some embodiments, the method may further comprise providing the estimating timing to one or more end users to coordinate patient room turnover.

In another aspect, the present disclosure provides methods for video collaboration. The method may comprise (a) obtaining a plurality of videos of a surgical procedure, wherein the plurality of videos are captured using a plurality of imaging devices; and (b) providing the plurality of videos to a plurality of end users, wherein each end user of the plurality of end users receives a different subset of the plurality of videos. In some embodiments, the different subsets of the plurality of videos may comprise one or more videos captured using different subsets of the plurality of imaging devices.

In another aspect, the present disclosure provides a method for video collaboration, the method comprising: (a) obtaining a plurality of videos of a surgical procedure; (b) determining an amount of progress for one or more steps of the surgical procedure based at least in part on the plurality of videos or a subset thereof; and (c) updating an estimated timing for performing or completing the one or more steps of the surgical procedure based at least in part on the amount of progress determined in step (b). In some embodiments, the method may further comprise providing the estimated timing to one or more end users to coordinate a performance or a completion of the surgical procedure or at least one other surgical procedure that is different than the surgical procedure. In some embodiments, the method may further comprise providing the estimated timing to one or more end users to coordinate patient room turnover. In some embodiments, the surgical procedure and the at least one other surgical procedure comprise two or more medical operations involving a donor subject and a recipient subject. In some embodiments, the method may further comprise scheduling or updating a scheduling for one or more other surgical procedure based on the estimated timing for performing or completing the one or more steps of the surgical procedure. In some embodiments, scheduling the one or more other surgical procedures comprises identifying or assigning an available time slot or an available operating room for the one or more other surgical procedures. In some embodiments, determining the amount of progress for the one or more steps of the surgical procedure comprises analyzing the plurality of videos to track a movement or a usage of one or more tools used to perform the one or more steps of the surgical procedure. In some embodiments, the estimated timing is derived from timing information associated with an actual time taken to perform a same or similar surgical procedure. In some embodiments, the method may further comprise generating a visual status bar based on the updated estimated timing, wherein the visual status bar indicates a total predicted time to complete the one or more steps of the surgical procedure. In some embodiments, the method may further comprise generating an alert or a notification when the estimated timing deviates from a predicted timing by a threshold value. In some embodiments, the threshold value is predetermined. In some embodiments, the threshold value is adjustable based on a type of procedure or a level of experience of an operator performing the surgical procedure. In some embodiments, the one or more end users comprise a medical operator, medical staff, medical vendors, or one or more robots configured to assist with or support the surgical procedure or at least one other surgical procedure. In some embodiments, the method may further comprise determining an efficiency of an operator performing the surgical procedure based at least in part on the updated estimated timing to complete the one or more steps of the surgical procedure. In some embodiments, the method may further comprise generating one or more recommendations for the operator to improve the operator's efficiency when performing a same or similar surgical procedure. In some embodiments, the method may further comprise generating a score or an assessment for the operator based on the operator's efficiency or performance of the surgical procedure.

In another aspect, the present disclosure provides a method for video collaboration, the method comprising: (a) obtaining a plurality of videos of a surgical procedure, wherein the plurality of videos are captured using a plurality of imaging devices; and (b) providing the plurality of videos to a plurality of end users, wherein at least one end user of the plurality of end users receives a different portion or subset of the plurality of videos than at least one other end user of the plurality of end users, based on an identity, an expertise, or an availability of the at least one end user. In some embodiments, the different subsets of the plurality of videos comprise one or more videos captured using different subsets of the plurality of imaging devices. In some embodiments, providing the plurality of videos comprises streaming or broadcasting the plurality of videos to the plurality of end users in real time as the plurality of videos are being captured by the plurality of imaging devices. In some embodiments, providing the plurality of videos comprises storing the plurality of videos on a server or storage medium for viewing or access by the plurality of end users. In some embodiments, providing the plurality of videos comprises providing a first video to a first end user and providing a second video to a second end user. In some embodiments, providing the plurality of videos comprises providing a first portion of a video to a first end user and providing a second portion of the video to a second end user. In some embodiments, the first video is captured using a first imaging device of the plurality of imaging devices and the second video is captured using a second imaging device of the plurality of imaging devices. In some embodiments, the second imaging device provides a different view of the surgical procedure than the first imaging device. In some embodiments, the second imaging device has a different position or orientation than the first imaging device relative to a subject of the surgical procedure or an operator performing one or more steps of the surgical procedure. In some embodiments, the first portion of the video corresponds to a different time point or a different step of the surgical procedure than the second portion of the video. In some embodiments, the method may further comprise providing the plurality of videos to the plurality of end users at one or more predetermined points in time. In some embodiments, the method may further comprise providing one or more user interfaces for the plurality of end users to view, modify, or annotate the plurality of videos. In some embodiments, the one or more user interfaces permit switching or toggling between two or more videos of the plurality of videos. In some embodiments, the one or more user interfaces permit viewing of two or more videos simultaneously. In some embodiments, the plurality of videos are stored or compiled in a video library, wherein providing the plurality of videos comprises broadcasting, streaming, or providing access to one or more of the plurality of videos through one or more video on demand services or models. In some embodiments, the method may further comprise implementing a virtual session for the plurality of end users to collaboratively view and provide one or more annotations for the plurality of videos in real time as the plurality of videos are being captured. In some embodiments, the one or more annotations comprise a visual marking or illustration provided by one or more of the plurality of end users. In some embodiments, the one or more annotations comprise audio, textual, or graphic commentary provided by one or more of the plurality of end users. In some embodiments, the virtual session permits the plurality of end users to modify a content of the plurality of videos. In some embodiments, modifying the content of the plurality of videos comprises adding or removing audio or visual effects.

In another aspect, the present disclosure provides a method for video collaboration, the method comprising: (a) providing one or more videos of a surgical procedure to a plurality of users; and (b) providing a virtual workspace for the plurality of users to collaborate based on the one or more videos, wherein the virtual workspace permits each of the plurality of users to (i) view the one or more videos or capture one or more recordings of the one or more videos, (ii) provide one or more telestrations to the one or videos or recordings, and (iii) distribute the one or more videos or recordings comprising the one or more telestrations to the plurality of users. In some embodiments, the virtual workspace permits the plurality of users to simultaneously stream the one or more videos and distribute the one or more videos or recordings comprising the one or more telestrations to the plurality of users. In some embodiments, the virtual workspace permits a first user to provide a first set of telestrations and a second user to provide a second set of telestrations simultaneously. In some embodiments, the virtual workspace permits a third user to simultaneously view the first set of telestrations and the second set of telestrations to compare or contrast inputs or guidance provided by the first user and the second user. In some embodiments, the first set of telestrations and the second set of telestrations correspond to a same video, a same recording, or a same portion of a video or a recording. In some embodiments, the first set of telestrations and the second set of telestrations correspond to different videos, different recordings, or different portions of a same video or recording. In some embodiments, the one or more videos comprise a highlight video of the surgical procedure, wherein the highlight video comprises a selection of one or more portions, stages, or steps of interest for the surgical procedure. In some embodiments, the first set of telestrations and the second set of telestrations are provided with respect to different videos or recordings captured by the first user and the second user. In some embodiments, the first set of telestrations and the second set of telestrations are provided or overlaid on top of each other with respect to a same video or recording captured by either the first user or the second user. In some embodiments, the virtual workspace permits each of the plurality of users to share one or more applications or windows at the same time with the plurality of users. In some embodiments, the virtual workspace permits the plurality of users to provide telestrations at the same time or modify the telestrations that are provided by one or more users at the same time. In some embodiments, the telestrations are provided on a live video stream of the surgical procedure or a recording of the surgical procedure.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A schematically illustrates an example of a video capture system for monitoring a surgical procedure, in accordance with some embodiments.

FIG. 1B schematically illustrates an example of a video capture system that is usable for video collaboration with a plurality of end users, in accordance with some embodiments.

FIG. 2 schematically illustrates a server configured to receive a plurality of videos captured by a plurality of imaging devices and transmit the plurality of videos to a plurality of end user devices, in accordance with some embodiments.

FIG. 3 schematically illustrates a direct transmission of a plurality of videos captured by a plurality of imaging devices to a plurality of end user devices, in accordance with some embodiments.

FIG. 4 schematically illustrates a user interface for viewing one or more videos captured by a plurality of imaging devices, in accordance with some embodiments.

FIG. 5 schematically illustrates a plurality of user interfaces configured to display different subsets of the plurality of videos to different end users, in accordance with some embodiments.

FIG. 6 schematically illustrates an example of a comparison between a timeline of predicted steps for a procedure and a timeline of the steps as they actually occur in real-time, in accordance with some embodiments.

FIG. 7 schematically illustrates various examples of different progress bars that may be displayed on a user interface based on an estimated timing to complete a surgical procedure, in accordance with some embodiments.

FIG. 8 schematically illustrates an example of an operating room schedule that may be updated based on estimated completion times for surgical procedures in different operating rooms, in accordance with some embodiments.

FIG. 9 schematically illustrates a donor surgery and a recipient surgery that may be coordinated using the methods and systems provided herein, in accordance with some embodiments.

FIG. 10 schematically illustrates one or more videos that may be provided to end users to view model examples for performing one or more steps of a surgical procedure, in accordance with some embodiments.

FIG. 11 schematically illustrates a computer system that is programmed or otherwise configured to implement methods provided herein.

FIGS. 12A, 12B, 12C, 12D, 12E, 12F, and 12G schematically illustrate various methods for streaming a plurality of videos to one or more end users, in accordance with some embodiments.

FIG. 13 schematically illustrates an example of a system for video collaboration, in accordance with some embodiments.

### DETAILED DESCRIPTION

The present disclosure provides methods and systems for video collaboration. The systems and methods of the present disclosure may enable medical practitioners in an operating room to selectively provide timely and accurate updates on the medical procedure to other individuals located remotely from the operating room. The systems and methods of the present disclosure may enable medical practitioners in an operating room to provide video data associated with one or more steps of a medical operation to one or more end users located outside of the operating room. The systems and methods of the present disclosure may also enable the sharing of different kinds of video data with different end users based on the relevancy of such video data to each end user. The systems and methods of the present disclosure may further enable the sharing of different kinds of video data to help coordinate parallel procedures (e.g., concurrent donor and recipient surgical procedures) and/or to help coordinate patient or operating room turnover in a medical facility such as a hospital.

In an aspect, the present disclosure provides methods for video collaboration. Video collaboration may involve using one or more videos to enhance communication or coordination between a first set of individuals and a second set of individuals. The first set of individuals may comprise one or more individuals who are performing or helping to perform a medical operation or surgical procedure. The second set of individuals may comprise one or more individuals who are located remote from a location where the medical operation or surgical procedure is being performed.

The video collaboration methods disclosed herein may be implemented using one or more videos obtained using one or more imaging devices that are configured to monitor a surgical procedure. Monitoring a surgical procedure may comprise tracking one or more steps of a surgical procedure based on a plurality of images or videos. In some cases, monitoring a surgical procedure may comprise estimating an amount of progress for a surgical procedure that is being performed based on a plurality of images or videos. In some cases, monitoring a surgical procedure may comprise estimating an amount of time needed to complete one or more steps of a surgical procedure based on a plurality of images or videos. In some cases, monitoring a surgical procedure may comprise evaluating a performance, a speed, an efficiency, or a skill of a medical operator performing the surgical procedure based on a plurality of images or videos. In some cases, monitoring a surgical procedure may comprise comparing an actual progress of a surgical procedure to an estimated timeline for performing or completing the surgical procedure based on a plurality of images or videos.

A surgical procedure may comprise a medical operation on a human or an animal. The medical operation may comprise one or more operations on an internal or external region of a human body or an animal. The medical operation may be performed using at least one or more medical products, medical tools, or medical instruments. Medical products, which may be interchangeably referred to herein as medical tools or medical instruments, may include devices that are used alone or in combination with other devices for therapeutic or diagnostic purposes. Medical products may be medical devices. Medical products may include any products that are used during an operation to perform the operation or facilitate the performance of the operation. Medical products may include tools, instruments, implants, prostheses, disposables, or any other apparatus, appliance, software, or materials that may be intended by the manufacturer to be used for human beings. Medical products may be used for diagnosis, monitoring, treatment, alleviation, or compensation for an injury or handicap. Medical products may be used for diagnosis, prevention, monitoring, treatment, or alleviation of disease. In some instances, medical products may be used for investigation, replacement, or modification of anatomy or of a physiological process. Some examples of medical products may range from surgical instruments (e.g., handheld or robotic), catheters, endoscopes, stents, pacemakers, artificial joints, spine stabilizers, disposable gloves, gauze, IV fluids, drugs, and so forth.

Examples of different types of surgical procedures may include but are not limited to thoracic surgery, orthopedic surgery, neurosurgery, ophthalmological surgery, plastic and reconstructive surgery, vascular surgery, hernia surgery, head and neck surgery, hand surgery, endocrine surgery, colon and rectal surgery, breast surgery, urologic surgery, gynecological surgery, and other types of surgery. In some cases, surgical procedures may comprise two or more medical operations involving a donor and a recipient. In such cases, the surgical procedures may comprise two or more concurrent medical operations to exchange biological material (e.g., organs, tissues, cells, etc.) between a donor and a recipient.

The systems and methods of the present disclosure may be implemented for one or more surgical procedures conducted in a health care facility. As used herein, a health care facility may refer to any type of facility, establishment, or organization that may provide some level of health care or assistance. In some examples, health care facilities may include hospitals, clinics, urgent care facilities, out-patient facilities, ambulatory surgical centers, nursing homes, hospice care, home care, rehabilitation centers, laboratory, imaging center, veterinary clinics, or any other types of facility that may provide care or assistance. A health care facility may or may not be provided primarily for short term care, or for long-term care. A health care facility may be open at all days and times, or may have limited hours during which it is open. A health care facility may or may not include specialized equipment to help deliver care. Care may be provided to individuals with chronic or acute conditions. A health care facility may employ the use of one or more health care providers (a.k.a. medical personnel/medical practitioner). Any description herein of a health care facility may refer to a hospital or any other type of health care facility, and vice versa.

In some cases, the health care facility may have one or more locations internal to the health care facility where one or more surgical operations may be performed. In some cases, the one or more locations may comprise one or more operating rooms. In some cases, the one or more operating rooms may only be accessible by qualified or approved individuals. Qualified or approved individuals may comprise individuals such as a medical patient or a medical subject undergoing a surgical procedure, medical operators performing one or more steps of a surgical procedure, and/or medical personnel or support staff who are supporting one or more aspects of the surgical procedure. For example, the medical personnel or support staff may be present in an operating room in order to help the medical operators perform one or more steps of the surgical procedure.

The methods of the present disclosure may comprise obtaining a plurality of videos of a surgical procedure. The plurality of videos may comprise one or more images of a surgical procedure. The plurality of videos may be obtained and/or used to monitor one or more aspects of the surgical procedure (e.g., a performance of one or more steps of a surgical procedure, a completion of one or more steps of a surgical procedure, a time elapsed, a time taken for each step of the surgical procedure, a time needed to complete one or more remaining steps in the surgical procedure, one or more movements or actions of a medical operator performing the surgical procedure, a use or an operation of one or more medical products or medical tools, etc.). In some cases, the plurality of videos may capture one or more viewpoints of a surgical site that a medical operator is operating on and/or one or more viewpoints of a surgical environment (e.g., an operating room) in which the surgical procedure is being performed.

The plurality of videos may be capturing using one or more imaging devices. The one or more imaging devices may comprise one or more imaging sensors, cameras, and/or video cameras. The one or more imaging devices may be configured to capture one or more images or videos of the surgical procedure. The one or more images or videos of the surgical procedure may include a patient or a subject of the surgical procedure, one or more medical personnel or medical operators assisting with the surgical procedure, and/or one or more medical products, medical tools, or medical instruments being used to perform or assist with the performance of the surgical procedure.

In some cases, the plurality of videos may be captured using a plurality of imaging devices. The plurality of imaging devices may comprise 1, 2, 3, 4, 5, 6, 7, 8, 9, 10, or more imaging devices. The plurality of imaging devices may comprise n imaging devices, where n is an integer that is greater than or equal to 2. The plurality of imaging devices may be provided in different positions and/or orientations relative to the subject or the medical operator performing the surgical operation on the subject.

The plurality of imaging devices may be provided in a plurality of different positions and/or orientations relative to a medical patient or subject undergoing a medical operation or a medical operator performing a medical operation. The plurality of imaging devices may be provided in a plurality of different positions and/or orientations relative to each other.

In some cases, the plurality of imaging devices may be attached to a ceiling, a wall, a floor, a structural element of an operating room (e.g., a beam), an operating table, a medical instrument, or a portion of a medical operator's body (e.g., the medical operator's hand, arm, or head). In some cases, the plurality of imaging devices may be releasably coupled to a ceiling, a wall, a floor, a structural element of an operating room, an operating table, a medical instrument, or a portion of a medical operator's body.

In some cases, the plurality of imaging devices may be movable relative to a surface or structural element on which the plurality of imaging devices are attached, fixed, or releasably coupled. For example, the plurality of imaging devices may be repositioned and/or rotated to adjust an imaging path of the plurality of imaging devices. In some cases, one or more joints, hinges, arms, rails, and/or tracks may be used to adjust a position and/or an orientation of the plurality of imaging devices. In some cases, the position and/or the orientation of each of the plurality of imaging devices may be manually adjustable by a human operator. In other cases, the position and/or the orientation of each of the plurality of imaging devices may be automatically adjustable in part based on computer-implemented optical tracking software. The position and/or the orientation of each of the plurality of imaging devices may be physically adjusted. The position and/or the orientation of each of the plurality of imaging devices may be adjusted or controlled remotely by a human operator.

The plurality of imaging devices may be configured to track and/or scan one or more areas or spaces in an operating room. The plurality of imaging devices may be configured to track and/or scan one or more areas or spaces in, on, or near a medical patient's or subject's body during a surgical operation. In some cases, each of the plurality of imaging devices may be configured to track and/or scan different areas or spaces.

The plurality of imaging devices may be configured to track and/or scan a movement of a medical operator. The plurality of imaging devices may be configured to track and/or scan the movements of a plurality of medical operators or medical personnel assisting the medical operator. In some cases, the plurality of imaging devices may be configured to track and/or scan the movements of different medical operators or medical personnel.

The plurality of imaging devices may be configured to track and/or scan a movement or a usage of medical device, instrument, or tool that is used for a medical procedure. The plurality of imaging devices may be configured to track and/or scan the movements or the usage of a plurality of medical devices, instruments, or tools that are used for a medical procedure. In some cases, each of the plurality of imaging devices may be configured to track and/or scan the movements or usage of different medical devices, instruments, or tools that are used during a medical procedure.

In some cases, the plurality of imaging devices may comprise one or more end user-specific imaging devices associated with a particular end user. The one or more end user-specific imaging devices may be configured to capture one or more videos for a certain type of end user and/or a particular end user to view. For example, a first imaging device may be configured to capture a first set of videos for a first type of end user (e.g., a family member of the medical patient) and a second imaging device may be configured to capture a second set of videos for a second type of end user (e.g., a medical operator who is not currently operating on the medical patient but is interested in tracking a progress of the surgical procedure). In some cases, the plurality of imaging devices may comprise one or more vendor-specific imaging devices associated with a particular vendor who provides, maintains, supports, and/or manages a particular medical device, instrument, or tool. The one or more vendor-specific imaging devices may be configured to capture one or more videos for a certain type of vendor and/or a particular vendor to view.

In some cases, the plurality of imaging devices may be configured to monitor and/or track a step of a surgical procedure or a plurality of steps of the surgical procedure. In some cases, each of the plurality of imaging devices may be configured to capture one or more videos of different steps in the surgical procedure. In such cases, the one or more videos captured for each of the different steps in the surgical procedure may be broadcast to and viewable by different end users.

Each of the plurality of imaging devices may have a set of imaging parameters associated with the operation and/or performance of the imaging devices. The imaging parameters may comprise an imaging resolution, a field of view, a depth of field, a frame capture speed, a sensor size, and/or a lens focal length. In some cases, two or more imaging devices of the plurality of imaging devices may have a same set of imaging parameters. In other cases, two or more imaging devices of the plurality of imaging devices may have different sets of imaging parameters.

In some embodiments, a single imaging device may be used to capture the plurality of videos. In some instances, multiple imaging devices may be used to capture the plurality of videos. In such cases, the multiple imaging devices may capture videos simultaneously.

As described above, the plurality of imaging devices may be configured to capture one or more videos of a surgical procedure, a medical operator performing the surgical procedure, medical personnel supporting the surgical procedure, or a subject undergoing a surgical procedure. In some cases, the plurality of imaging devices may be configured to capture one or more videos of one or more steps of a surgical procedure as the one or more steps are being performed. In some cases, the plurality of imaging devices may be configured to capture one or more videos of one or more tools used to perform each step of the surgical procedure.

The plurality of videos captured by the imaging devices or a subset of the plurality of videos captured by the imaging devices may be processed and/or analyzed by a video processing module. The video processing module may be configured to analyze one or more videos captured by the imaging devices after one or more steps of a surgical procedure are completed. Alternatively, the video processing module may be configured to analyze one or more videos captured by the imaging devices while one or more steps of a surgical procedure are being performed.

In some embodiments, one or more videos from a single imaging device may be analyzed by the video processing module. Alternatively, one or more videos captured by multiple imaging devices may be analyzed together. In such cases, timing information between the various imaging devices may be synchronized in order to get a sense of comparative timing between the videos captured by each of the imaging devices. For instance, each imaging device may have an associated clock or may communicate with a clock. Such clocks may be synchronized with one another in order to accurately determine the timing of the videos captured by multiple imaging devices. In some instances, multiple imaging devices may communicate with a single clock. In some instances, the timing on the clocks may be different, but a disparity between the clocks can be known. The disparity between the clocks can be used to ensure that the videos being analyzed from multiple imaging device are synchronized or are using the proper timing.

In some cases, the video processing module may be configured to determine a type of surgical procedure based on the plurality of videos captured by the imaging devices. In some cases, the video processing module may be configured to determine the type of surgical procedure based on the tools used, medical personnel present, type of patient, steps taken, and/or time taken for each step of the surgical procedure.

In some cases, the video processing module may be configured to recognize one or more steps in a surgical procedure as a medical operator performs the surgical procedure. In some cases, the one or more steps in the surgical procedure may be recognized in part based on the type of surgical procedure. In other cases, the one or more steps in the surgical procedure may be recognized in part based on the tools used by the medical personnel and/or actions taken by the medical personnel. Object recognition may be utilized to recognize the tools used and/or steps taken by the medical personnel. For example, if the first step is to make an incision, the plurality of videos may be analyzed to recognize that the first step is being performed when a scalpel is used to make the incision. In some cases, the motion of a medical personnel and/or medical personnel's hands to make an incision may be used to recognize that the first step is being performed.

The video processing module may be configured to generate a set of predicted steps corresponding to one or more remaining steps of the surgical procedure. The set of predicted steps may be derived or estimated in part based on the type of surgical procedure. The set of predicted steps may be derived or estimated in part based on the various steps performed or completed by a medical operator. In some embodiments, the video processing module may be configured to use video information, audio data, medical records, and/or inputs by medical personnel, alone or in combination, to predict one or more remaining steps to be performed by a medical operator.

In some cases, the video processing module may be configured to update a step list for a surgical procedure in real-time. This may help medical personnel to track or monitor a progress of the surgical procedure as the medical personnel performs or completes one or more steps of the surgical procedure. In some cases, a visual indicator (e.g., checkmark, highlight, different color, icon, strikethrough, underline) may be provided to visually differentiate completed steps from steps that have not yet been completed. In some instances, detected steps or conditions during the medical procedure may cause the predicted or recommended steps to change. The video analysis system may automatically detect when such a condition has occurred.

The video processing module may be configured to predict or estimate a timing to perform or complete one or more steps of a surgical procedure. In some cases, the predicted timing for one or more steps of a surgical procedure may vary based on different anatomy types. For example, particular anatomies may make certain steps within the procedure more difficult, which may result in more time being taken for that particular step. For instance, Step 1 may take longer with Anatomy Type B than Anatomy Type A. Similarly, Step 2 may take longer with Anatomy Type B than with Anatomy Type A. However, Step 4 may take about the same amount of time regardless of whether the patient is of Anatomy Type A or Anatomy Type B. To account for such variances, the video processing module may be configured to detect, determine, or recognize an anatomy type of a medical patient and to adjust the predicted timing for one or more steps of a surgical procedure based on the detected anatomy type. The recognition of certain portions of the patient's body, such as the various specific features within the portion of the patient's body, may be useful to detect the steps needed to perform a surgical procedure and/or to predict the timings to perform or complete each step of the surgical procedure.

In some cases, the video processing module may be configured to predict or estimate a timing to perform or complete one or more steps of a surgical procedure based on one or more medical devices and/or products identified or recognized from video images. In some instances, certain types of medical devices or tools may be required for a particular surgical procedure, and the identity of the tools and/or devices used may be useful in detecting the steps to be performed and the timings associated with those steps.

In some instances, the video processing module may be configured to predict or estimate a timing to perform or complete one or more steps of a surgical procedure based on audio information. For instance, medical personnel may announce the step that he or she is about to perform before taking the step, or while performing the step. In some instances, medical personnel may dictate their actions as they are being performed. Optionally, the medical personnel may ask for assistance or tools from other medical personnel, which may provide information that may be useful for detecting the steps being performed and predicting the timings associated with those steps.

In some embodiments, the video processing module may be configured to recognize when medical personnel has performed or completed each step. In some embodiments, when the system detects that the medical personnel has performed or completed a step (or sub-step), the estimated timing for one or more subsequent steps may be updated. In some cases, as each step is completed, a checkmark or other type of visual indicator may be displayed on a step list associated with the surgical procedure to visually distinguish a completed step from one or more remaining steps of the surgical procedure. In some instances, when a step is completed, the completed step may visually disappear from the step list associated with the surgical procedure.

In some cases, the plurality of videos may be processed and/or analyzed by the video processing module to derive timing information associated with a performance or a completion of one or more steps of the surgical procedure. As one or more steps are being performed, the timing information associated with the one or more steps of the surgical procedure may be recorded and measured by the video processing module. For instance, as the video processing module detects each step is starting, the system may make note of the time at which each step is occurring. In some cases, the video processing module may be configured to recognize a time at which various steps are started and a length of time it takes for the steps to be completed.

In some cases, the video processing module may be configured to derive a total timing information associated with the entire surgical procedure. For instance, alternative to or in addition to showing timing information for each step, the overall timing information or progress may be displayed. For instance, a total amount of time that the medical personnel is lagging, or ahead of the predicted time, may be displayed. In some instances, the total amount of time may be displayed as a numerical time value (e.g., hours, minutes, seconds), or as a relative value (e.g., percentage of predicted time or actual time, etc.). In some instances, a visual display, such as a status bar may be provided. The visual display may include a status bar representing a timeline. In some cases, the status bar may show a total predicted time to complete the medical procedure. The predicted breakdown of times at each step may or may not be shown on the status bar. The medical personnel's current amount of time spent may be shown relative to the status bar. An updated predicted amount of time to complete the medical procedure may also be displayed as a second status bar or overlap with the first status bar. Overall timing or progress information may be provided to the medical personnel in a visual manner.

As described elsewhere herein, the video processing module may be configured to determine an actual time taken to complete or perform one or more steps of a surgical procedure. The video processing module may be further configured to determine or predict an estimated amount of time needed to complete or perform one or more remaining steps of a surgical procedure.

In some embodiments, the video processing module may be configured to compare the actual time needed to complete one or more steps of a surgical procedure against an estimated or predicted time in order to determine if a medical operator is ahead of schedule and/or behind schedule. The estimated or predicted time may correspond to an actual time taken to perform one or more similar steps in another surgical procedure (e.g., a same or similar surgical procedure previously performed before the current surgical procedure). A comparison of the predicted time and the actual amount of time to complete the step may be presented. In some embodiments, the comparison may be provided as numbers, fractions, percentages, visual bars, icons, colors, line graphs, or any other type of comparison.

The video processing module may be configured to compare the predicted timing for one or more steps with the actual timing for the various steps as they occur in real-time. When a significant disparity in timing exists, the disparity may be flagged. In some instances, a notification may be provided to a medical personnel in real-time while they are performing the procedure. For instance, a visual notification or audio notification may be provided when the disparity has been detected.

In some embodiments, a disparity may need to reach a threshold in order to be flagged. The threshold for the disparity may be set ahead of time. The threshold may be set based on an absolute value (e.g., number of minutes, seconds, etc.) and/or relative value (e.g., percentage of the predicted time for the step). In some instances, the threshold value may depend on the standard deviation from the various data sets collected. For example, if a wider variation in timing is provided through the various data sets, then a greater threshold or tolerance may be provided. The threshold value may be fixed or may be adjustable (e.g., based on a type of surgical procedure or a level of experience of the surgeon performing the surgical procedure). In some embodiments, the medical personnel, or another individual at a health care facility (e.g., colleague, supervisor, administrator) may set the value. In some embodiments, a single threshold may be provided. Alternatively, multiple levels of thresholds may be provided. The multiple threshold levels may be useful in determining a degree of disparity and may result in different types of actions or notifications to the medical personnel. In some cases, an alert or notification may be generated if a threshold disparity is met or exceeded.

In some cases, the video processing module may be configured to compare a predicted timing of one or more steps of a surgical procedure to an actual timing of the one or more steps of the surgical procedure. For example, a first step of the surgical procedure may have a particular predicted timing, and the first step may actually be performed within approximately the same amount of time as predicted. This may cause no flags to be raised. In another example, a second step of the surgical procedure may be expected to occur within a particular length of time, but in practice may actually take a significantly longer period of time. When a significant deviation occurs, this difference may be flagged. This may allow medical personnel to later review this disparity and figure out why the step took longer than expected. This may be useful for introducing new techniques or providing feedback to the medical personnel on how the medical personnel may be able to perform more efficiently in the future.

In some cases, the video processing module may be configured to detect a difference in timing between the predicted amount of time for the step and the actual amount of time taken for the step. When the difference in timing between the predicted amount of time for the step and the actual amount of time taken for the step exceeds a threshold, as described elsewhere herein, the portion of the video corresponding to the step may automatically be flagged as relevant.

In some cases, it may be desirable to flag a step as relevant when it takes much longer than predicted. A medical personnel or other individual may wish to review the step and determine why it took so much longer than predicted. In some instances, a step taking longer may be indicative of an event or issue that arose that required more time for the medical personnel to perform the step. In some instances, the step taking longer may indicate that the medical personnel is not using the most efficient technique, or is having difficulty with a particular step, which may be helpful to provide additional review.

In some cases, it may be desirable to flag a step as relevant when it takes significantly less time than predicted. A medical personnel or other individual may wish to review the step and see how the medical personnel was able to save time on a particular step. This may provide a useful teaching opportunity to other individuals that may wish to mimic a similar technique. This may also provide a recognition of a particular skillset that the medical personnel may have. In some instances, a medical personnel may be able to perform a step faster than predicted. When this occurs, it may be useful information to provide for educational purposes to other medical personnel. This information may be flagged as a useful teaching opportunity to other medical personnel.

Even if the steps that are performed match up, the video may be analyzed to detect if there is significant deviation from expected timing of the step. For example, it may be expected that step 1 typically takes about 5 minutes to perform. If the step ends up taking 15 minutes, this difference in timing may be recognized and/or flagged. When a significant difference in time is provided, a message (e.g., visual and/or audio message) may optionally be provided to the medical personnel. For instance, if a step is taking longer than expected, a display may show information that may aid the medical personnel in performing the step. Helpful hints or suggestions may be provided in real-time. In some embodiments, the timing information may be tracked in order to update a prediction of a timing of the surgery. In some instances, updates to expected timing and/or the percentage of completion of a procedure may be provided to a medical personnel while the medical personnel is performing the procedure.

In some embodiments, the degree of discrepancy for timing before flagging the discrepancy may be adjustable. For instance, if an average step takes about 15 minutes, but the medical personnel takes 16 minutes to perform the step, the degree of discrepancy may not be sufficient enough to make a note or raise a flag. In some instances, the degree of discrepancy needed to raise a flag may be predetermined. In some instances, the degree of discrepancy to reach a threshold to raise a flag may be on an absolute time scale (e.g., number of minutes, number of seconds). In some instances, the degree of discrepancy to reach a threshold to raise a flag may be on a relative time scale (e.g., percentage of amount of time that a step typically takes). The threshold value may be fixed, or may be adjustable. In some embodiments, a medical personnel may provide a preferred threshold (e.g., if the discrepancy exceeds more than 5 minutes, or more than 20% of expected procedure time). In other embodiments, the threshold may be set by an administrator of a health care facility, or another group member or medical operator that supervises or works with the medical personnel.

In some cases, the video processing module may be configured to determine if the medical operator is conducting or performing a step that is different than a predicted step. For example, if the medical personnel is expected to open a vessel, but the medical personnel instead performs a different step, such a difference may be flagged. In some instances, a visual or audio indicator may be provided to the medical personnel as soon as the disparity is detected. For example, a message may be displayed on the screen indicating that the medical personnel is deviating from the plan. The message may include an indication of the predicted step and/or the actual detected step occurring. Optionally, an audio message may provide similar information. For example, an audio message may indicate a deviation has been detected from the predicted step. An indication of the details of the predicted step and/or detected deviation may or may not be provided. Such feedback may be provided in real-time while the medical operator is performing the procedure. This may advantageously allow medical personnel to assess progress and make any corrections or adjustments if necessary.

In some cases, the video processing module may be configured to determine an efficiency of a medical operator as the medical operator is performing one or more steps of the surgical procedure in real time. In some cases, the video processing module may be configured to determine which steps took longer than initially estimated. In some cases, the video processing module may be configured to determine if the medical operator made any mistakes or deviations from a standard procedure that decreased an efficiency of the medical operator.

In some embodiments, the video processing module may automatically provide feedback to the medical personnel regarding the execution of the procedure. For instance, the video processing module may automatically indicate if significant deviations in steps and/or timing occurred. In some instances, the video processing module may provide recommendations to the medical personnel on differences that can be made by the medical personnel to improve efficiency and/or effectiveness of the procedure. Optionally, a score or assessment may be provided for the medical personnel's completion of the procedure.

The plurality of videos and/or the information derived from the plurality of videos by the video processing module may be provided to one or more end users. The one or more end users may comprise a subject of the surgical procedure, a medical operator of the surgical procedure (e.g., a doctor, a surgeon, or a physician), one or more friends or family members of the subject, medical personnel outside of an operating room in which the surgical procedure is performed, medical support staff, medical vendors, medical students, medical staff being trained (e.g., interns or residents), other medical operators outside of the operating room (e.g., medical operators who will be performing one or more steps in the surgical procedure, medical operators who have already completed one or more steps in the surgical procedure, or medical operators who are operating on another patient or subject in parallel in the case of donor and recipient surgical procedures), or medical staff who are helping to coordinate the scheduling and use of the operating room in which the surgical procedure is being performed.

In some cases, the plurality of videos and/or the information derived from the plurality of videos may be provided to one or more medical devices. The plurality of videos and/or the information derived from the plurality of videos may be displayed or consumed by third party medical devices to perform additional operations and/or to support one or more steps of a surgical procedure. In one example, the plurality of videos and/or the information derived from the plurality of videos may be provided to one or more robots or nanorobots in real time (e.g., as the plurality of videos are being captured, or as information is being derived or generated from the plurality of videos). The one or more robots or nanorobots may be configured to receive the plurality of videos and any information derived from the plurality of videos in real time, and to use the plurality of videos or the information derived from the plurality of videos to perform one or more steps of a surgical procedure.

In some cases, the end users may comprise one or more medical vendors. Medical vendors may include individuals or entities that may provide support before, during, or after a medical procedure. Medical vendors may also include outside medical professionals or specialists, consultants, technicians, manufacturers, financial support, social workers, or any other individuals. In some cases, medical vendors may comprise individuals or entities who provide medical equipment (e.g., medical products, medical devices, or medical tools and instruments). In some cases, vendors may be entities, such as companies, that manufacture and/or distribute medical products. The vendors may have representatives that may be able to provide support to personnel using the medical devices. The vendor representatives (who may also be known as product specialists or device reps), may be knowledgeable about one or more particular medical products. Vendor representatives may aid medical personnel (e.g., surgeons, surgical assistants, physicians, nurses) with any questions they may have about the medical products. Vendor representatives may aid in selection of sizing or different models of particular medical products. Vendor representatives may aid in function of medical products. Vendor representatives may help a medical personnel use product, or troubleshoot any issues that may arise. These questions may arise in real-time as the medical personnel are using a product.

The plurality of videos may be provided to a plurality of end users. In some cases, each end user of the plurality of end users may receive a different subset of the plurality of videos. In some cases, each end user of the plurality of end users may receive one or more videos captured using different imaging devices.

In some cases, the surgical procedure may be performed in a first location. The plurality of videos may be captured using one or more imaging devices located in or near the first location. The plurality of videos may be provided to one or more end users located in a second location that is different than the first location. In some cases, the plurality of videos may be provided to one or more end users located in the first location.

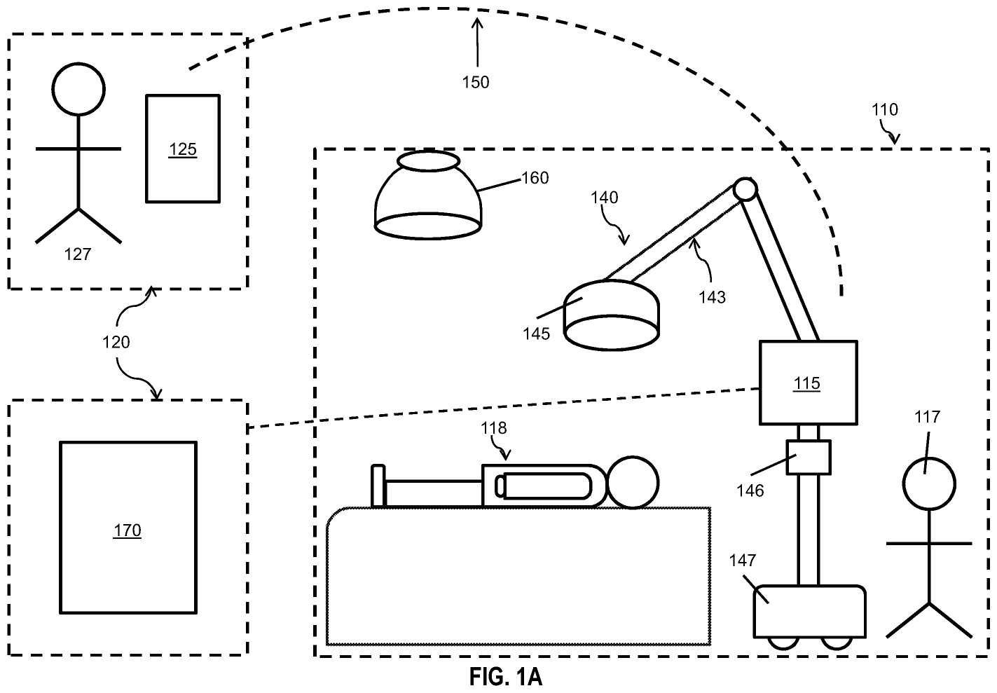

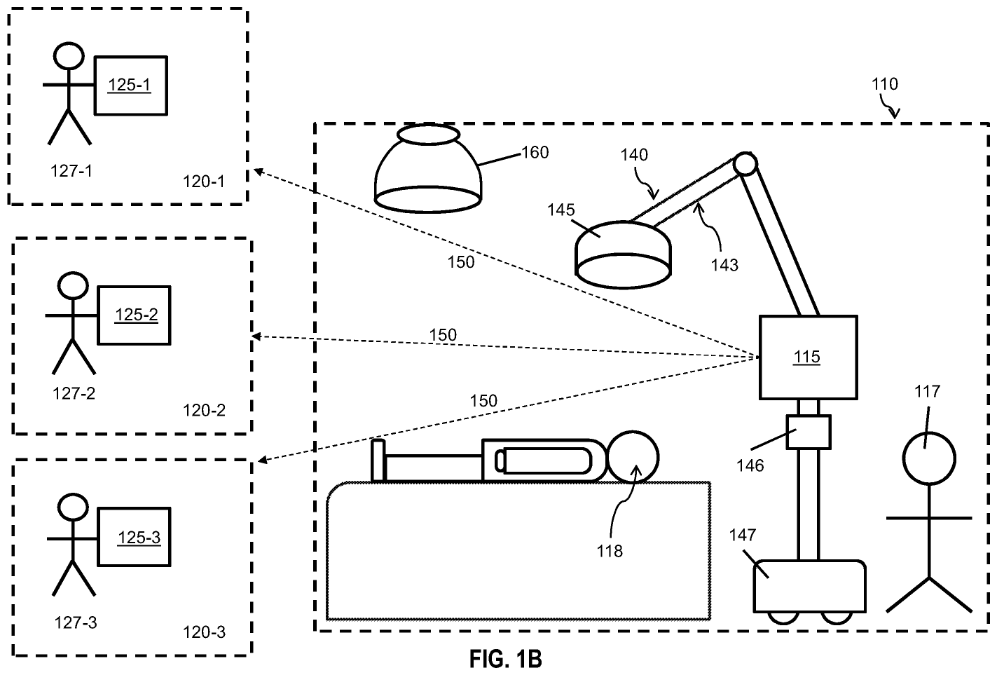

FIG. 1A and FIG. 1B show examples of a video capture system utilized within a medical suite, such as an operating room. The video capture system may comprise the one or more imaging devices described above. The video capture system may be configured to capture images or videos of a surgical procedure, a surgical site, or an operating environment in which a surgical procedure is being performed.

The video capture system may allow for communications between the medical suite and one or more end users or remote individuals, in accordance with embodiments of the invention. Communication may optionally be provided between a first location 110 and a second location 120. In some cases, the video capture system may also comprise a local communication device 115. In some cases, the local communication device 115 may be operably coupled to the one or more imaging devices described above. The local communication device 115 may optionally communicate with a remote communication device 125.

As shown in FIG. 1B, in some cases the local communication device 115 may communicate with a plurality of remote communication devices 125-1, 125-2, and 125-3. The plurality of videos captured by the plurality of imaging devices may be provided to the plurality of remote communication devices 125-1, 125-2, and 125-3. The plurality of remote communication devices 125-1, 125-2, and 125-3 may be located in a plurality of locations 120-1, 120-2, and 120-3 that are remote from the first location 110 where a surgical procedure is being performed. The plurality of remote communication devices 125-1, 125-2, and 125-3 may be associated with different end users 127-1, 127-2, and 127-3. In some cases, the different end users 127-1, 127-2, and 127-3 may comprise vendors or vendor representatives who may be able to provide remote support during one or more steps of a surgical procedure.

The first location 110 may be a medical suite, such as an operating room of a health care facility. A medical suite may be within a clinic room or any other portion of a health care facility. A health care facility may be any type of facility or organization that may provide some level of health care or assistance. In some examples, health care facilities may include hospitals, clinics, urgent care facilities, out-patient facilities, ambulatory surgical centers, nursing homes, hospice care, home care, rehabilitation centers, laboratory, imaging center, veterinary clinics, or any other types of facility that may provide care or assistance. A health care facility may or may not be provided primarily for short term care, or for long-term care. A health care facility may be open at all days and times, or may have limited hours during which it is open. A health care facility may or may not include specialized equipment to help deliver care. Care may be provided to individuals with chronic or acute conditions. A health care facility may employ the use of one or more health care providers (a.k.a. medical personnel/medical practitioner). Any description herein of a health care facility may refer to a hospital or any other type of health care facility, and vice versa.

The first location may be any room or region within a health care facility. For example, the first location may be an operating room, surgical suite, clinic room, triage center, emergency room, or any other location. The first location may be within a region of a room or an entirety of a room. The first location may be any location where an operation may occur, where surgery may take place, where a medical procedure may occur, and/or where a medical product is used. In one example, the first location may be an operating room with a patient 118 that is being operated on, and one or more medical personnel 117, such as a surgeon or surgical assistant that is performing the operation, or aiding in performing the operation. Medical personnel may include any individuals who are performing the medical procedure or aiding in performing the medical procedure. Medical personnel may include individuals who provide support for the medical procedure. For example, the medical personnel may include a surgeon performing a surgery, a nurse, an anesthesiologist, and so forth. Examples of medical personnel may include physicians (e.g., surgeons, anesthesiologists, radiologists, internists, residents, oncologists, hematologists, cardiologists, etc.), nurses (e.g., CNRA, operating room nurse, circulating nurse), physicians' assistants, surgical techs, and so forth. Medical personnel may include individuals who are present for the medical procedure and authorized to be present.

A second location 120 may be any location where an end user 127 is located. The second location may be remote to the first location. For instance, if the first location is a hospital, the second location may be outside the hospital. In some instances, the first and second locations may be within the same building but in different rooms, floors, or wings. The second location may be at an office of the end user or remote individual. A second location may be at a residence of an end user or remote individual.

In some embodiments, medical personnel in the first location 110 may communicate with one or more remote individuals or end users in the second location 120. The medical personnel in the first location 110 may use a local communication device 115 to communicate with the end users in the second location 120. An end user or remote individual may have a remote communication device 125 which may communicate with the local communication device 115 at the first location. Any form of communication channel 150 may be formed between the remote communication device and the local communication device. The communication channel may be a direct communication channel or indirect communication channel. The communication channel may employ wired communications, wireless communications, or both. The communications may occur over a network, such as a local area network (LAN), wide area network (WAN) such as the Internet, or any form of telecommunications network (e.g., cellular service network). Communications employed may include, but are not limited to 3G, 4G, LTE communications, and/or Bluetooth, infrared, radio, or other communications. Communications may optionally be aided by routers, satellites, towers, and/or wires. The communications may or may not utilize existing communication networks at the first location and/or second location.

Communications between the remote communication devices and the local communication devices may be encrypted. Optionally, only authorized and authenticated remote communication devices and local communication devices may be able to communicate over a communication system.

In some embodiments, a remote communication device and/or local communication device may communicate with one another through a communication system. The communication system may facilitate the connection between the remote communication device and the local communication device. The communication system may aid in accessing scheduling information at a health care facility. The communication system may aid in presenting, on a remote communication device, a user interface for an end user or remote individual to monitor a surgical procedure being performed in the first location.

In some cases, the one or more imaging devices may be integrated with a communication device (e.g., the local communication device or the remote communication device). Alternatively, the one or more imaging devices may be operatively coupled to the local communication device or the remote communication device. The one or more imaging devices may face a user (e.g., a medical operator in the first location or an end user in the second location) when the user looks at a display of the communication device. The one or more imaging devices may face away from a user when the user looks at a display of the communication device. In some instances, multiple imaging devices may be provided which may face in different directions. The imaging devices may be capable of capturing images and/or videos at a desired resolution. For instance, the imaging devices may be capable of capturing images and/or videos in one or more display resolutions, including Standard Definition (SD), High Definition (HD), Full High Definition (FHD), Widescreen Ultra Extended Graphics Array (WUXGA), 2K, Quad High Definition (QHD), Wide Quad High Definition (WQHD), Ultra High Definition (UHD), 4K, 8K, or any resolution greater than or less than 8K. In some cases, the imaging devices may be configured to capture images and/or videos with a resolution of 640×360 pixels, 720×480 pixels, 960×540 pixels, 1280×720 pixels, 1280×1080 pixels, 1600×900 pixels, 1920×1080 pixels, 2048×1080 pixels, 2160×1080 pixels, 2560×1080 pixels, 2560×1440 pixels, 3200×1800 pixels, 3440×1440 pixels, 3840×1080 pixels, 3840×1600 pixels, 840×2160 pixels, 4096×2160 pixels, 5120×2160 pixels, 5120×2880 pixels, 7680×4320 pixels, 160×120 pixels, 240×160 pixels, 320×240 pixels, 400×240 pixels, 480×320 pixels, 640×480 pixels, 768×480 pixels, 854×480 pixels, 800×600 pixels, 960×640 pixels, 1024×576 pixels, 1024×600 pixels, 1024×768 pixels, 1366×768 pixels, 1366×768 pixels, 1360×768 pixels, 1280×800 pixels, 1152×864 pixels, 1440×900 pixels, 1280×1024 pixels, 1400×1050 pixels, 1680×1050 pixels, 1600×1200 pixels, 1920×1200 pixels, 2048×1152 pixels, 2048×1536 pixels, 2560×1600 pixels, 2560×2048 pixels, 3200×2048 pixels, 3200×2400 pixels, 3840×2400 pixels, or any resolution with N×M pixels, where N and M are integers greater than or equal to 1. In some cases, the imaging devices may be configured to capture images and/or videos with an aspect ratio of 4:3, 16:9, 16:10, 18:9, or 21:9. An imaging device on a remote communication device may capture an image of an end user in the second location. An imaging device on a local communication device may capture an image of a medical personnel in the first location. An imaging device on a local communication device may capture an image of a surgical site and/or medical tools, instruments or products in the first location.

The communication device (e.g., the local communication device or the remote communication device) may comprise one or more microphones or speakers. A microphone may capture audible sounds such as the voice of a user. For instance, the remote communication device microphone may capture the speech of an end user in the second location and a local communication device microphone may capture the speech of a medical personnel in the first location. One or more speakers may be provided to play sound. For instance, a speaker on a remote communication device may allow an end user in the second location to hear sounds captured by a local communication device in the first location, and vice versa. In some embodiments, an audio enhancement module may be provided. The audio enhancement module may be supported by a video capture system. The audio enhancement module may comprise an array of microphones that may be configured to clearly capture voices within a noisy room while minimizing or reducing background noise. The audio enhancement module may be separable or may be integral to the video capture system.

In some cases, the communication device (e.g., the local communication device or the remote communication device) may comprise a display screen. The display screen may be a touchscreen. The display screen may accept inputs by a user's touch, such as finger. The display screen may accept inputs by a stylus or other tool.

In some cases, the communication device (e.g., the local communication device or the remote communication device) may be any type of device capable of communication. For instance, the communication device may be a smartphone, tablet, laptop, desktop, server, personal digital assistant, wearable (e.g., smartwatch, glasses, etc.), or any other type of device.

In some embodiments, the local communication device 115 may be supported by a medical console 140. The local communication device may be permanently attached to the medical console, or may be removable from the medical console. In some instances, the local communication device may remain functional while removed from the medical console. The medical console may optionally provide power to the local communication device when the local communication device is attached to (e.g., docked with) the medical console. The medical console may be mobile console that may move from location to location. For instance, the medical console may include wheels that may allow the medical console to be wheeled from location to location. The wheels may be locked into place at desired locations. The medical device may optionally comprise a lower rack and/or support base 147. The lower rack and/or support base may house one or more components, such as communication components, power components, auxiliary inputs, and/or processors.

In some cases, the medical console may optionally include one or more cameras 145, 146. In some cases, the one or more cameras may be positioned on a distal end of an articulating arm 143 of the medical console. The cameras may be capable of capturing images of the patient 118, or portion of the patient (e.g., surgical site). The cameras may be capable of capturing images of the medical devices. The cameras may be capable of capturing images of the medical devices as they rest on a tray, or when they are handled by a medical personnel and/or used at the surgical site. The cameras may be capable of capturing images at any resolution, such as those described elsewhere herein. The cameras may be used to capture a still images and/or video images. The cameras may be capturing images in real time.

In some cases, one or more of the cameras may be movable relative to the medical console. For instance, one or more cameras may be supported by an arm 143. The arm may include one or more sections. In one example, a camera may be supported at or near an end of an arm. The arm may include one or more sections, two or more section, three or more sections, four or more sections, or more sections. The sections may move relative to one another or a body of the medical console. The sections may pivot about one or more hinges or joints. In some embodiments, the movements may be limited to a single plane, such as a horizontal plane. Alternatively, the movements need not be limited to a single plane. The sections may move horizontally and/or vertically. A camera may have at least one, two, three, or more degrees of freedom. An arm may optionally include a handle that may allow a user to manually manipulate the arm to a desired position. The arm may remain in a position to which it has been manipulated. A user may or may not need to lock an arm to maintain its position. This may provide a steady support for a camera. The arm may be unlocked and/or re-manipulated to new positions as needed. In some embodiments, a remote user may be able to control the position of the arm and/or cameras.

In some cases, the cameras and/or imaging sensors of the present disclosure may be provided separately from and independent of the medical console or one or more displays. The cameras and/or imaging sensors may be used to capture images and/or videos of an ongoing surgical procedure or a surgical site that is being operated on, that has been operated on, or that will be operated on as part of a surgical procedure. In some cases, the cameras and/or imaging sensors disclosed herein may be used to capture images and/or videos of a surgeon, a doctor, or a medical worker assisting with or performing one or more steps of the surgical procedure. The cameras and/or imaging sensors may be moved independently of the medical console or one or more displays. For instance, the cameras and/or imaging sensors may be positioned and/or oriented in a first direction or towards a first region, and the medical console or the one or more displays may be positioned and/or oriented in a second direction or towards a second region. In some cases, the one or more displays may be moved independently of the one or more cameras and/or imaging sensors without affecting or changing a position and/or orientation of the cameras or imaging sensors. The one or more displays described herein may be used to display the images and/or videos captured using the cameras and/or imaging sensors. In some cases, the one or more displays may be used to display images, videos, or other information or data provided by a remote vendor representative to one or more medical workers in a healthcare facility or an operating room where a surgical procedure may be performed or conducted. The images or videos displayed on the one or more displays may comprise an image or a video of a vendor representative. The images or videos displayed on the one or more displays may comprise images and/or videos of the vendor representative as the vendor representative provides live feedback, instructions, guidance, counseling, or demonstrations. Such live feedback, instructions, guidance, counseling, or demonstrations may relate to a usage of one or more medical instruments or tools, or a performance of one or more steps in a surgical procedure using the one or more medical instruments or tools.

In some embodiments, the one or more cameras and/or imaging sensors may comprise two or more cameras and/or imaging sensors. The two or more cameras and/or imaging sensors may be moved independently of each other. In some cases, a first camera and/or imaging sensor may be movable independently of and relative to a second camera and/or imaging sensor. In some cases, the second camera and/or imaging sensor may be fixed or stationary. In other cases, the second camera and/or imaging sensor may be movable independently of and relative to the first camera and/or imaging sensor.

FIG. 13 schematically illustrates an example of a system 1300 that may be used for video collaboration. The system 1300 may comprise a medical console 1301, one or more cameras 1310, and at least one display unit 1320. The medical console 1301 may comprise one or more components or features that enable the at least one display unit 1320 and the one or more cameras 1310 to be moved independently of and relative to each other. The one or more components or features may comprise, for example, an arm or a movable element that provides one or more degrees of freedom. In some cases, the one or more cameras 1310 may be moved independently of each other to capture different views of an ongoing surgical procedure. In some cases, the one or more cameras 1310 may be moved independently of the at least one display unit 1320 without affecting or changing a position and/or an orientation of the at least one display unit 1320. In some cases, the at least one display unit 1320 may be moved independently of the one or more cameras 1310 without affecting or changing a position and/or orientation of the one or more cameras 1310.

In some embodiments, one or more cameras may be provided at or near the first location. The one or more cameras may or may not be supported by the medical console. In some embodiments, one or more cameras may be supported by a ceiling 160, wall, furniture, or other items at the first location. For instance, one or more cameras may be mounted on a wall, ceiling, or other device. Such cameras may be directly mounted to a surface, or may be mounted on a boom or arm. For instance, an arm may extend down from a ceiling while supporting a camera. In another example, an arm may be attached to a patient's bed or surface while supporting a camera. In some instances, a camera may be worn by medical personnel. For instance, a camera may be worn on a headband, wrist-band, torso, or any other portion of the medical personnel. A camera may be part of a medical device or may be supported by a medical device (e.g., endoscope, etc.). The one or more cameras may be fixed cameras or movable cameras. The one or more cameras may be capable of rotating about one or more, two or more, or three or more axes. The one or more cameras may include pan-tilt-zoom cameras. The cameras may be manually moved by an individual at the first location. The cameras may be locked into position and/or unlocked to be moved. In some instances, the one or more cameras may be remotely controlled by one or more remote users. The cameras may zoom in and/or out. Any of the cameras may have any of the resolution values as provided herein. The cameras may optionally have a light source that may illuminate an area of interest. Alternatively, the cameras may rely on external light sources.

The plurality of images and/or videos captured by the one or more cameras 145, 146 may be analyzed using a video processing module as described elsewhere herein. The video may able analyzed in real-time. The videos may be sent to a remote communication device. This may allow a remote use to remotely view images or videos captured by the field of view of the cameras or imaging devices located at or near the first location. For instance, the remote user may view the surgical site and/or any medical devices being used at the first location. The remote user may be able to view the medical personnel in the first location. The remote user may be able to view these in substantially real-time. For instance, this may be within 1 minutes or less, 30 seconds or less, 20 seconds or less, 15 seconds or less, 10 seconds or less, 5 seconds or less, 3 seconds or less, 2 seconds or less, or 1 second or less of an event actually occurring. This may allow a remote user to monitor the surgical procedure at the first location without needing to be physically at the first location. The medical console and cameras may aid in providing the remote user with the necessary images, videos, and/or information to have a virtual presence at the first location.

The video analysis may occur locally at the first location 110. In some embodiments, the analysis may occur on-board a medical console 140. For instance, the analysis may occur with aid of one or more processors of a communication device 115 or another computer that may be located at the medical console. In some instances, the video analysis may occur remotely from the first location. In some instances, one or more servers 170 may be utilized to perform video analysis. The server may be able to access and/or receive information from multiple locations and may collect large datasets. The large datasets may be used in conjunction with machine learning in order to provide increasingly accurate video analysis. Any description herein of a server may also apply to any type of cloud computing infrastructure. The analysis may occur remotely, and feedback may be communicated back to the console and/or location communication device in substantially real-time. Any description herein of real-time may include any action that may occur within a short span of time (e.g., within less than or equal to about 10 minutes, 5 minutes, 3 minutes, 2 minutes, 1 minute, 30 seconds, 20 seconds, 15 seconds, 10 seconds, 5 seconds, 3 seconds, 2 seconds, 1 second, 0.5 seconds, 0.1 seconds, 0.05 seconds, 0.01 seconds, or less).

In some cases, the plurality of videos captured by the plurality of imaging devices may be saved to one or more files for viewing at a later time (e.g., after the surgical procedure is completed). The one or more files may be stored in a server. The server may be located remote from a location in which the surgical procedure is performed. The server may comprise a cloud server. The one or more files stored on the server may be accessible by one or more end users during and/or after a surgical procedure. The one or more end users may be located remote from the location in which the surgical procedure is performed.

In some cases, the plurality of videos may be broadcasted and/or streamed to a plurality of end user devices. The plurality of end user devices may comprise one or more remote communication devices as described elsewhere herein. The plurality of remote communication devices may be configured to display at least a subset of the plurality of videos to one or more end users. The plurality of videos may be streamed in real time to one or more end users. The plurality of videos may be broadcasted and/or streamed from a first location to a second location. The first location may correspond to a location in which the surgical procedure is performed. The second location may correspond to another location that is remote from the first location. The plurality of videos may be streamed, broadcasted, and/or shared with one or more end users via a communications network as shown in FIG. 1A. In some cases, the plurality of videos may be temporarily stored on a server or a cloud server before the plurality of videos are streamed and/or broadcasted to one or more end users. In some cases, the plurality of videos may be processed and/or analyzed by a video processing module before the plurality of videos are streamed and/or broadcasted to one or more end users. The video processing module may be provided on a remote server or a cloud server. In some cases, the video processing module may be provided on a computing device that is located in an operating room, medical suite, or health care facility in which the surgical procedure is performed.

The plurality of videos may be saved or stored on a server before the plurality of videos are provided to the one or more end users via streaming, live broadcasting, or video on demand. The server may be located in a first location where the surgical procedure is performed. The server may be located in a second location that is remote from the first location in which the surgical procedure is performed. In some cases, the plurality of videos may be transmitted from the server to one or more remote end users using a communications network.

In some cases, the plurality of videos may be streamed or broadcasted directly from the plurality of imaging devices to one or more end users. In such cases, the plurality of videos may be transmitted from the plurality of imaging devices to one or more communication devices of one or more end users via a communications network.

In some cases, the plurality of videos may be viewed using a display unit that is operably coupled to the plurality of imaging devices. The display unit may be located in the operating room where the surgical procedure is performed. In some cases, the display unit may be located in another room within the health care facility in which the surgical procedure is performed (e.g., another operating room or a patient waiting room).

The plurality of end users may receive and/or view the plurality of videos or a subset thereof on one or more remote communication devices. The one or more remote communication devices may be configured to receive the plurality of videos via a communications network. The one or more remote communication devices may be configured to display the plurality of videos or a subset thereof to one or more end users. The one or more remote communication devices may comprise a computer, a desktop, a laptop, and/or a mobile device of one or more end users. The one or more end users may use the one or more remote communication devices to view at least a subset of the plurality of videos.

In some instances, the video may be displayed to one or more end users outside the location of the medical personnel (e.g., outside the operating room, or outside the health care facility). In some instances, the video may be displayed to one or more end users (e.g., other medical practitioners, vendor representatives) that may be providing support to the medical procedure remotely. In some instances, the video may be broadcast to a number of end users who are interested in monitoring, tracking, or viewing one or more steps of a surgical procedure. The end users may be viewing the surgical procedure for training or evaluation purposes. The videos as live-streamed to the one or more end users may automatically have the data anonymized. The personal information may be removed in real-time so that no end users outside the operating room may view any personal information of the individual.

In some cases, the plurality of videos may be viewed and played back at a later time. In some instances, when a video is provided at a later time, the personal information may automatically be removed and/or anonymized.

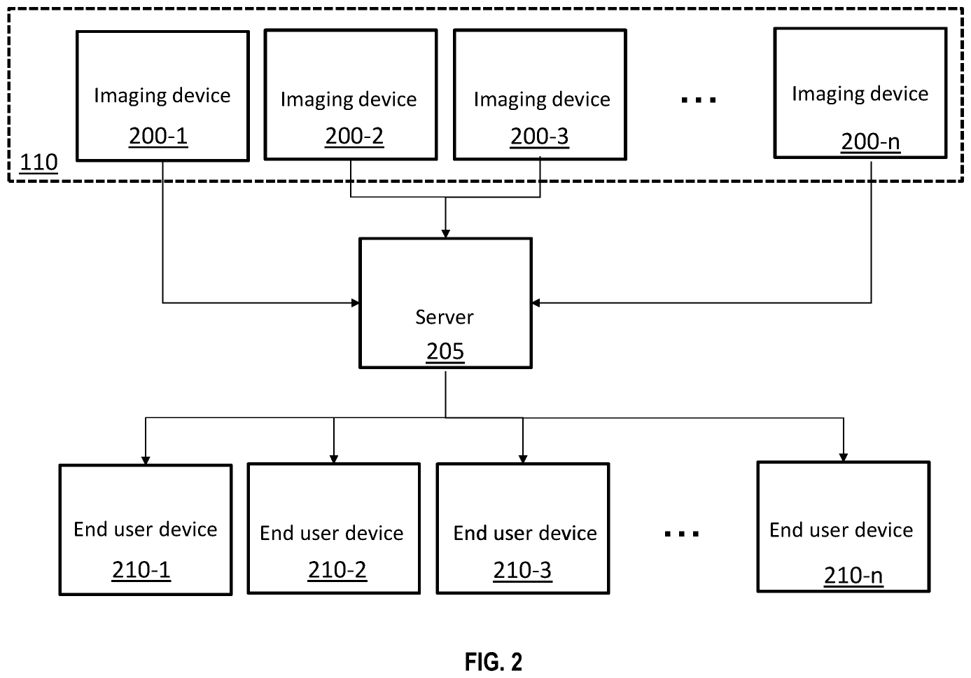

FIG. 2 illustrates a plurality of imaging devices comprising one or more imaging devices 200-1, 200-2, and 200-3 that are in communication with a server 205. The plurality of imaging devices 200-n may comprise n number of imaging devices, where n is greater than or equal to 1. The server 205 may be configured to receive a plurality of videos captured by the plurality of imaging devices 200-n and to transmit the plurality of videos to a plurality of end user devices. The plurality of end user devices 210-n may comprise one or more end user devices 210-1, 210-2, 210-3, and so on. The plurality of end user devices 210-n may comprise n number of end user devices, where n is greater than or equal to 1. In some cases, the server 205 may comprise a video processing module as described above. The video processing module may be configured to analyze the plurality of videos received from the plurality of imaging devices 200-n before the plurality of videos are transmitted to the plurality of end user devices 210-n. The plurality of imaging devices 200-n may be located in a first location 110 as described above. The plurality of end user devices 210-n may be located in one or more remote locations that are remote from the first location 110. In some cases, the one or more remote locations may correspond to different locations outside the first location but within a same health care facility in which the first location is located. For example, the one or more remote locations may correspond to different operating rooms that are remote from an operating room in which a surgical procedure is being performed. Alternatively, the one or more remote locations may correspond to different rooms (e.g., a waiting room, a ward, a consulting room, a day room, an emergency room, a pharmacy, an intensive care unit, etc.) that are remote from an operating room in which a surgical procedure is being performed. In some cases, the one or more remote locations may correspond to one or more locations outside the health care facility in which the first location is located. In some cases, each of the plurality of end user devices may be located in a plurality of different locations that are remote from the first location. For example, a first end user device may be in a second location, a second end user device may be in a third location, a third end user device may be in a fourth location, and so on.

In any of the embodiments described herein, a plurality of end users located in one or more remote locations may utilize one or more end user devices to independently or collectively provide remote support to medical personnel in the first location. In any of the embodiments described herein, a plurality of end users located in one or more remote locations may utilize one or more end user devices to interact, communicate and/or collaborate with each other. In any of the embodiments described herein, a plurality of end users located in one or more remote locations may utilize one or more end user devices to collectively interact, communicate and/or collaborate with medical personnel in the first location. In any of the embodiments described herein, a plurality of end users located in one or more remote locations may utilize one or more end user devices to independently interact, communicate and/or collaborate with medical personnel in the first location.

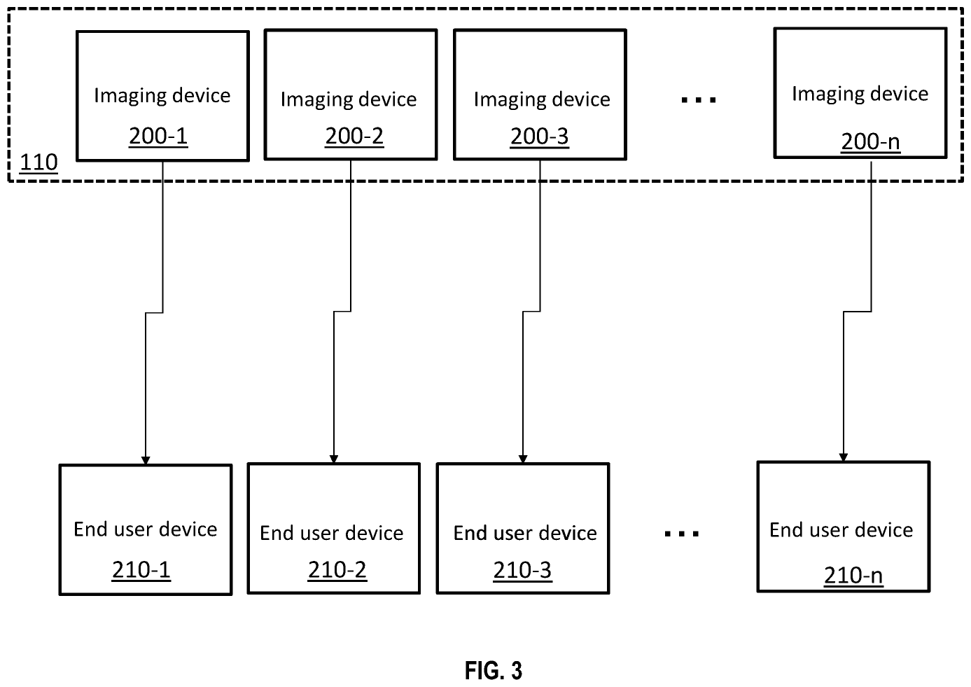

FIG. 3 illustrates a plurality of imaging devices 200-n comprising one or more imaging devices 200-1, 200-2, 200-3, and so on that are in communication with a plurality of end user devices 210-n comprising one or more end user devices 210-1, 210-2, 210-3, and so on. The plurality of imaging devices 200-n may be located in a first location 110 as described above. The plurality of end user devices 210-n may be located in one or more remote locations that are remote from the first location 110. As described above, the one or more remote locations may correspond to different remote locations that are remote from each other and/or remote from the first location. In some cases, the plurality of imaging devices 200-n may be configured to directly transmit the plurality of videos captured by the imaging devices to the plurality of end user devices 210-n using a communications network. In other cases, the plurality of imaging devices 200-n may be operatively coupled to one or more local communication devices that are located in or near the first location 110 where a surgical procedure is being performed. In such cases, the one or more local communication devices may be configured to directly transmit the plurality of videos captured by the imaging devices to the plurality of end user devices 210-n using any one or more communications networks described herein.

The plurality of videos may be viewed through a user interface that is displayed on a communication device. In some cases, the communication device may comprise a local communication device on a medical console located in an operating room in which the surgical procedure is performed. In one example, the user interface may be displayed on a screen of a device at the location of the medical personnel performing the procedure. In other cases, the user interface may be displayed on a screen (e.g., a touch screen) of a remote communication device of an end user. The remote communication device may comprise a computer, a desktop, a laptop, and/or a mobile device of an end user. In some cases, the remote communication device may comprise an end user device that is configured to receive one or more videos captured using the plurality of imaging devices.

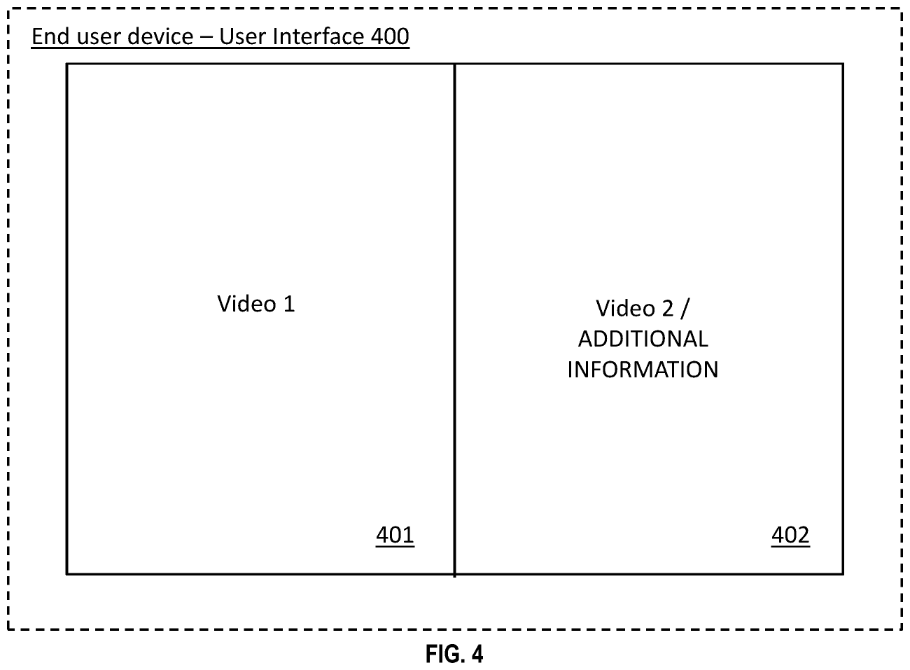

The user interface may present one or more visual representations of a medical procedure being performed. As shown in FIG. 4, in some cases, the user interface 400 may be configured to display multiple regions 401 and 402 which show information about one or more medical procedures. The regions may be configured to display at least one video of the plurality of videos captured by the plurality of imaging devices. The regions may include icons, images, videos, text, or interactive buttons. In some instances, the various regions may include additional information, such as the associated health care facility, the associated location at the health care facility (e.g., operating room), medical personnel (e.g., surgeon's name), type of procedure (e.g., procedure code, procedure name), timing information associated with one or more steps of the surgical procedure, and/or medical product information (e.g., identification of medical product being used). The user interface 400 may be displayed on a device of an end user.

In some instances, the various regions may be viewable when the medical procedure is taking place or scheduled to take place. The regions may be displayed at one or more predetermined times. The one or more predetermined times may be associated with an identity or a type of an end user. The regions may also be tied to the communication system so that if one end user is able to see the region, the region is no longer displayed to a second end user. This allows each end user to view only relevant portions or steps of a surgical procedure for a given time.

In some instances, only a single region may be displayed on an end user's screen. Optionally, the end user's access to one or more regions may be reserved or dedicated to one or more portions or steps of a medical procedure. The end user's may only be presented with the single relevant region for the live procedure at a given time.

In some embodiments, multiple end users may see a same region for one or more steps of a medical procedure. For example, if a particular step of the surgical procedure is relevant or of interest to a plurality of end users, there may be multiple end users who are able to view the region at the same time.

In some instances, the user interface may be configured to display an option for an end user to specify whether the end user wishes to access the communication system by procedure step or by time. The user may be prompted to select an option.

The user interface may show any number of views of one or more steps of a surgical procedure. In some embodiments, the user interface may show any number of views of a surgical site and/or a product to be used. These views may be stationary and/or movable as needed. The number of views and/or types of views may change as needed. An end user may be able to control the view. For example, an end user may be able to zoom in or out of one or more regions of the user interface as needed. For example, an end user may manipulate the images or videos on the end user's screen to zoom in or out, or to expand, reduce, or resize a particular view.

In some instances, auxiliary images from devices connected to be the console may be presented. For instance, images from ECG devices, endoscopes, laparoscopes, ultrasound devices, or any other devices may also be viewable. The images may be of sufficient resolution so that the medical personnel can provide effective support. The user interface may allow an end user to view a relevant medical procedure and/or product and provide support as needed.

In some cases, a user interface may optionally show other data. For example, readouts from one or more medical devices may be displayed. For example, a patient's electroencephalogram (EEG), electrocardiogram (ECG/EKG), electromyogram (EMG), heartrate, oxygenation level, or other data may be shown. Personal information about a patient, such as a patient's name, patient ID number, patient's demographics, patient's address, patient's medical history, may or may not be made available to view by the vendor representative

In some instances, an end user may manipulate the user interface to toggle between multiple views. For instance, if the end user needs to provide more focused attention to a particular procedure, the end user may zoom in or allow the information pertaining to that procedure to expand and take up more space on the screen or the entirety of the screen.

The user interface may display images or videos captured by one or more image capturing devices and provide support to an end user (e.g., a medical practitioner) in real-time, in accordance with embodiments of the invention. The user interface may be displayed on a communication device such as a local communication device on a medical console. The user interface may be displayed on a screen of a device at the location of the medical personnel performing the procedure. The user interface may be displayed on a screen of a remote communication device of an end user.

In some instances, a single image or video may be displayed to an end user at a given moment. The end user may toggle between different views from different cameras. The images may be displayed in a sequential manner.

In some other instances, multiple images or videos may be simultaneously displayed to one or more end users at a given moment. For example, multiple images or videos may be displayed in a row, in a column, and in/or in an array. Images or videos may be simultaneously displayed in a side-by-side manner. In some instances, smaller images or videos may be inserted within a bigger image or video. A user may select the smaller image to expand the smaller image or video and shrink the larger image or video. Any number of images or videos may be displayed simultaneously. For instance, two or more, three or more, four or more, five or more, six or more, eight or more, ten or more, or twenty or more images or videos may be displayed simultaneously. The various displayed images or videos may include images or videos from different imaging devices. The imaging devices may be configured to live-stream different videos to different regions of the user interface simultaneously.

In some instances, the videos from the multiple imaging device may be provided in a side by side view or an array view. The images from the multiple imaging devices may be shown simultaneously on the same screen.

In some cases, an end user may use the user interface to mark or flag a portion of a video as relevant. When a portion of a video is flagged as relevant, the videos from all of the imaging devices that were captured at the same time may be brought up and shown together. In other instances, only the video from the imaging device that has been flagged as relevant may be brought up and shown. In some embodiments, the video analysis system may select the imaging device that provides the best view of the procedure for a given time period that has been flagged as relevant. This may be the same imaging device that provided the video that has been flagged as relevant, or another imaging device.

In some cases, the user interface may include a display of additional information. The additional information may relate to a procedure being performed or about to be performed by medical personnel. The additional information may include steps relating to the medical procedure. For example, a list of steps predicted in order to perform the medical procedure may be displayed. The list of steps may be presented in a chronological order with the first step appearing at the top of the list. In some embodiments, a single list of steps may be presented. In some embodiments, the lists may have sub-lists and so forth. For instance, the lists may appear in a nested fashion, where a step may correspond to a second list having details of how to perform each step. Any number of layers of lists and sub-lists for each step may be presented.

In some cases, the user interface may be configured to display one or more videos simultaneously. The one or more videos may be provided in a side-by-side configuration. In some cases, the user interface may be configured to permit an end user to toggle between one or more videos, or to enlarge a first video relative to a second video.

In some cases, the user interface may be configured to display different videos or different views of a surgical procedure based on an amount of progress, a number of steps performed, a number of steps remaining, an amount of time elapsed, and/or an amount of time remaining.

The user interface may be configured to provide additional data corresponding to the surgical procedure or the plurality of videos displayed within the user interface. For example, the user interface may be configured to display additional data from an EKG/ECG or one or more sensors for monitoring a heart rate, a blood pressure, an oxygen saturation, a respiration, and/or a temperature of a subject undergoing a surgical procedure. In some cases, the additional data may be overlaid over a portion of one or more videos displayed within the user interface. The user interface may be configured to provide real time updates of the additional data during the surgical procedure.

In some cases, each remote communication device of the plurality of end users may be configured to display an end user-specific user interface. The end user-specific user interface may comprise an individualized or customized user interface that is tailored for each end user. The individualized or customized user interface may allow each end user to view only the videos that are relevant to the end user. For instance, a vendor may only see a subset of the plurality of videos that are relevant to the vendor, such as one or more videos in which a tool provided by the vendor is used. Further, a doctor or a medical operator may see a different subset of the plurality of videos, and a family member or a friend of the medical subject undergoing the surgical procedure may see another different subset of the plurality of videos.

In some cases, the user interface may be configured to anonymize personal information or personal data that may be captured and/or displayed in one or more videos of the plurality of videos. In such cases, the user interface may be configured to strip out or redact the personal information or personal data that may be linked to the patient undergoing a surgical procedure.

In some embodiments, the user interface may have personal data removed. In some cases, images or videos captured by the plurality of imaging devices may include personal information relating to the patient. For instance, a chart or document may have a patient's name, birth date, social security number, address, telephone number, insurance information, or any other type of personal information. In some embodiments, it may be desirable to redact personal information for the patient from the videos. In some instances, it may be desirable to anonymize information shown on the videos in order to comply with one or more set of rules, procedures or laws. In some instances, all information shown on the videos may be compliant with the Health Insurance Portability and Accountability Act (HIPAA-compliant).

In some cases, the user interface may show information relating to a patient, such as a chart or a set of medical records. The chart or medical records may include a physical document and/or electronic documents that have been accessed during the procedure. The information may include personal or sensitive information relating to the patient. Such information may be automatically identified by the video analysis system. The video analysis system may use object and/or character recognition to be able to identify information displayed. In some instances, word recognition techniques may be used to analyze the information. Natural language processing (NLP) algorithms may optionally be used. In some instances, when personal information is identified, the personal information may be automatically removed. Any description herein of personal information may include any sensitive information relating to the patient, or any information that may identify or provide personal characteristics of the patient.

The user interface may be configured to display one or more images or videos captured by one or more imaging devices as described elsewhere herein. In some instances, one or more of the images or videos may include personal information that may need to be removed. In some instances, an identifying characteristic on a patient may be captured by a video camera (e.g., the patient's face, medical bracelet, etc.). The one or more images may be analyzed to automatically detect when the identifying characteristic is captured within the image and remove the identifying characteristic. In some instances, object recognition may be used to identify personal information. For instance, recognition of an individual's face or medical bracelet may be employed in order to identify personal information that is to be removed. In some instances, a patient's chart or medical records may be captured by the video camera. The personal information on the patient's chart or medical records may be automatically detected and removed.

The personal information may be removed by being redacted, deleted, covered, obfuscated, or using any other techniques that may conceal the personal information. In some instances, the systems and methods provided herein may be able to identify the size and/or shape of the information displayed that needs to be removed. A corresponding size and/or shape of the redaction may be provided. In some instances, a mask may be provided over the image to cover the personal information. The mask may have the corresponding shape and/or size.

Accordingly, any video that is recorded and/or displayed may anonymize the personal information of the patient. In some instances, the video that is displayed at the location of the medical personnel (e.g., within the operating room) may show all of the information without redacting the personal information in real-time. Alternatively, the video that is displayed at the location of the medical personnel may have the personal information removed.

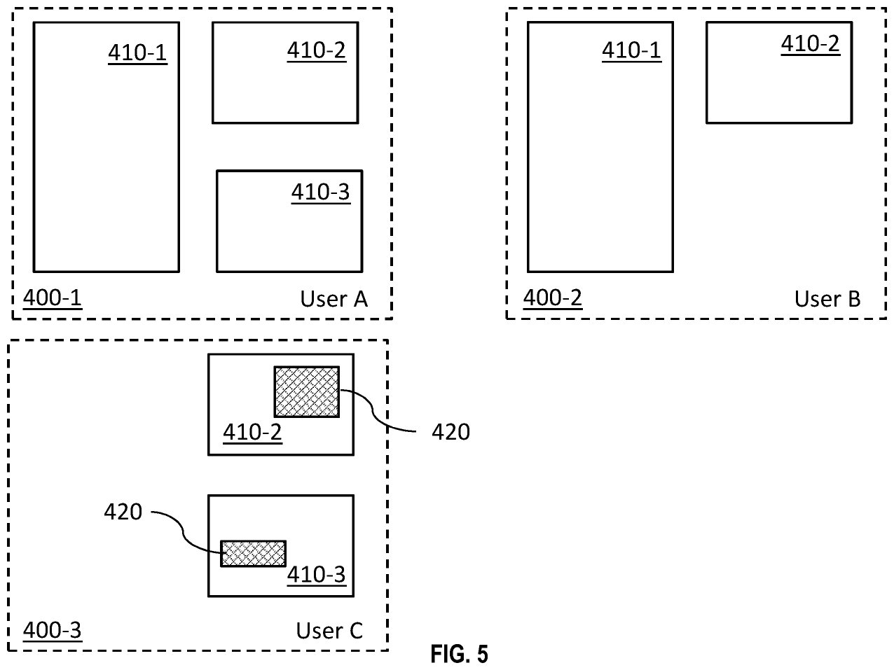

FIG. 5 illustrates a plurality of different user interfaces 400-1, 400-2, and 400-3 that may be displayed on different end user devices associated with different end users. The plurality of different user interfaces 400-1, 400-2, and 400-3 may be configured to display different videos or different subsets of plurality of videos captured by the plurality of imaging devices. For example, a first end user (User A) may see a first user interface 400-1 that is configured to display a first set of videos 410-1, 410-2, and 410-3. In some cases, a second end user (User B) may see a second user interface 400-2 that is configured to display a different second set of videos 410-1 and 410-2. In some cases, a third end user (User C) may see a third user interface 400-3 that is configured to display a different third set of videos 410-2 and 410-3. In some cases, portions of the videos may be redacted to remove personal information. For example, one or more portions of the plurality of videos 410-2 and 410-3 viewable by User C may be redacted 420 to cover, hide, or block personal information associated with the medical patient undergoing a surgical procedure. The plurality of user interfaces illustrated in FIG. 5 may be configured or customized for any number of different end users and/or any collaborative application of the video collaboration systems and methods described herein. The plurality of user interfaces may be configured or customized depending on which videos or subsets of videos are shared with one or more end users. The plurality of user interfaces may comprise different layouts if different videos or different subsets of videos are shared with the one or more end users. The plurality of user interfaces may display different videos or different subsets of videos for different end users based on a type of end user, an identity of an end user, a relevance of one or more videos to an end user, and/or whether an end user is allowed to or qualified to view one or more videos.

In some cases, the plurality of videos may be provided to one or more end users via a communications network. The plurality of videos may be provided to one or more end users by live streaming in real time while one or more steps of a surgical procedure are being performed. In some cases, the plurality of videos may be provided to one or more end users as videos that may be accessible and viewable by the one or more end users after one or more steps of a surgical procedure have been performed or completed.

In some cases, the one or more end users may receive a same set of videos captured by the plurality of imaging devices. In other cases, each end user of the plurality of end users may receive a different subset of the plurality of videos. In some cases, each end user may receive one or more videos based on a type of end user, an identity of an end user, a relevance of one or more videos to an end user, and/or whether an end user is allowed to or qualified to view one or more videos. In some cases, each end user may receive different videos or different subsets of videos based on a type of end user, an identity of an end user, a relevance of one or more videos to an end user, and/or whether an end user is allowed to or qualified to view one or more videos. The different subsets of the plurality of videos may comprise one or more videos captured using different subsets of the plurality of imaging devices.

Each end user of the plurality of end users may receive a different subset of the plurality of videos based on a relevance of a particular subset of the plurality of videos to each end user. In some cases, the one or more end users may receive different videos or different subsets of videos that correspond to a particular aspect or portion of a surgical procedure for which the one or more end users may be able to provide guidance or remote support. In some cases, each end user may receive one or more videos that relate to an interest of each end user. For example, each end user may receive one or more videos that capture a particular viewpoint of interest of the surgical procedure. In another example, each end user may receive one or more videos that capture different steps of interest for a surgical procedure. In some cases, a first end user may receive a first video of a first step of the surgical procedure, a second end user may receive a second video of a second step of the surgical procedure, and so on. In some cases, each end user may receive one or more videos that capture different tools being used during the surgical procedure. In some cases, a first end user may receive a first video of a first medical tool being used during the surgical procedure, a second end user may receive a second video of a second medical tool being used during the surgical procedure, and so on. In some cases, the one or more end users may receive different videos or different subsets of videos depending on whether an end user is allowed to or qualified to view one or more videos. In some cases, the one or more end users may receive different videos or different subsets of videos depending on one or more regulations or laws such as the Health Insurance Portability and Accountability Act. In some cases, the one or more end users may receive different videos or different subsets of videos depending on one or more rules set by the medical patient, the surgical operator, an administrator or member of the health care facility, a friend or family member of the medical patient, and/or any other end user as described herein. The one or more rules may dictate which end users may view and/or access a particular set or subset of videos captured by the one or more imaging devices, and the conditions under which such videos may be viewed or accessed. In some cases, the one or more end users may receive different parts or sections of the same video or video frame depending on a set of rules associated with the viewability and/or accessibility of the plurality of videos, a specialty or a role of the one or more end users, or a relevance of the different parts or sections of the video or video frame to each of the one or more end users. In some cases, one or more parallel streams from a console or a broadcaster may be provided to applicable or authorized end users. The one or more parallel streams may be configured to provide each end user with different videos or video compositions depending on the set of rules associated with the viewability and/or accessibility of the plurality of videos, a specialty or a role of the one or more end users, or a relevance of different parts or sections of a video or video frame to each end user.

In some cases, the plurality of videos may be provided to one or more medical vendors. In such cases, each of the one or more medical vendors may view one or more subsets of the plurality of videos. The one or more subsets may comprise one or more videos that track a usage of a tool provided by the vendor during a surgical procedure. In some cases, the one or more videos may track a portion or a step of a surgical procedure during which the vendor's support, input, or guidance may be needed. In such cases, each vendor may receive different subsets of the plurality of videos which correspond to a usage of one or more medical tools or instruments provided, supported, or managed by the vendor.

In some cases, each of the plurality of end users may receive different subsets of the plurality of videos at different times or for different steps of the surgical procedure. For example, a first end user may receive a first subset of the plurality of videos at a first point in time during the surgical procedure, and a second end user may receive a second subset of the plurality of videos at a second point in time during the surgical procedure. In some cases, a first end user may view a first subset of the plurality of videos during a first time period, and a second end user may view a second subset of the plurality of videos at a second time period that is different than the first time period. In some cases, the first time period and the second time period may overlap. In other cases, the first time period and the second time period may not or need not overlap. The first time period may correspond to a first step of the surgical procedure. The second time period may correspond to a second step of the surgical procedure.

In some cases, a plurality of end users may view different videos concurrently or simultaneously. For example, a first end user may view a video of one or more steps of the surgical procedure from a first view point, and a second end user may view a video of one or more steps of the surgical procedure from a second view point that is different than the first view point.

In some cases, one or more end users may receive and/or view each of the plurality of videos captured by the plurality of imaging devices. For example, friends and/or family members of a medical subject undergoing a surgical procedure may be able to view each and every video captured by the plurality of imaging devices. In such cases, the friends and/or family members may be able to monitor each step of the surgical procedure from every viewpoint captured by the plurality of imaging devices. In some cases, the friends and/or family members may be able to toggle between different videos to view one or more steps of the surgical procedure from a plurality of different viewpoints. In some cases, the friends and/or family members may be able to view at least a subset of the plurality of videos simultaneously in order to monitor different viewpoints of the surgical procedure concurrently.

In some cases, a medical operator may be able to receive and/or view each of the plurality of videos captured by the plurality of imaging devices after completing the surgical procedure. In such cases, the medical operator may view different portions of the surgical procedure in order to evaluate a skill or an efficiency of the medical operator when performing different steps of the surgical procedure.

In some cases, medical support staff may be able to receive and/or view each of the plurality of videos captured by the plurality of imaging devices while the surgical procedure is being performed. In such cases, the medical support staff may be able to use the plurality of videos to determine how long the surgical procedure might take, coordinate scheduling of other surgical procedures, book or reserve different operating rooms if a surgical procedure is taking longer than expected, adjust operating room assignments, or to notify other medical operators of a progress of a surgical procedure or an estimated time to complete the surgical procedure. Alternatively, the medical support staff may be able to use the plurality of videos to determine what medical instruments or tools need to be prepared for subsequent steps of the surgical procedure.

In some cases, each of the plurality of videos may be provided to other medical operators who will be operating on a medical subject in another step of the surgical procedure. In such cases, the other medical operators may be able to monitor one or more steps of procedure preceding and/or leading up to a step of the procedure during which they will be operating on the medical subject. The other medical operators may use the plurality of videos to prepare for their turn.

In some cases, the plurality of imaging devices may be used to coordinate two or more parallel (i.e., concurrent) surgical procedures. The two or more parallel procedures may comprise a first surgical procedure on a first subject and a second surgical procedure on a second subject. The first subject may comprise a donor patient and the second subject may comprise a recipient patient. Alternatively, the first subject may comprise a recipient patient and the second subject may comprise a donor patient. In such cases, the plurality of imaging devices may be configured to capture one or more videos of the first surgical procedure and/or the second surgical procedure. The one or more videos may be provided to a first medical operator for the first surgical procedure and/or a second medical operator the second surgical procedure. The one or more videos may be provided to at least one of the first medical operator or the second medical operator so that the first medical operator and the second medical operator may coordinate a timing of the first surgical procedure and the second surgical procedure and minimize standby time between the completion of one or more steps for the first surgical operation and one or more steps for the second surgical operation.

In some cases, the plurality of videos may be selectively distributed to one or more end users using an artificial intelligence module. The artificial intelligence module may be configured to implement one or more algorithms to determine, in real time, which videos or subsets of videos are viewable and/or accessible by each end user as the one or more videos are being captured the plurality of imaging devices. The artificial intelligence module may be configured to implement one or more algorithms to determine, in real time, which videos or subsets of videos are viewable and/or accessible by each end user as one or more steps of a surgical procedure are being performed. The artificial intelligence module may be configured to determine, in real time, which videos or subsets of videos are viewable and/or accessible by each end user based on an identity of each end user, a role of each end user in supporting the surgical procedure, a type of support being provided by each end user, a relevance of one or more videos to each end user, and/or whether each end user is allowed to or qualified to view one or more videos.

In some cases, the plurality of videos captured by the plurality of imaging devices may be provided to one or more end users to help the one or more end users estimate or predict one or more timing parameters associated with an ongoing surgical procedure. The one or more timing parameters may comprise information such as an amount of time elapsed since the start of the surgical procedure, an estimated amount of time to complete the surgical procedure, a number of steps completed since the start of the surgical procedure, a number of steps remaining to complete the surgical procedure, an amount of progress for the surgical procedure, a current step of the surgical procedure, and/or one or more remaining steps in the surgical procedure. In some cases, the one or more timing parameters may comprise and/or correspond to timing information associated with one or more steps of a surgical procedure as described elsewhere herein. In some cases, a video processing module may be configured to analyze and/or process the plurality of videos captured by the plurality of imaging devices to determine the one or more timing parameters.

In some cases, the one or more timing parameters may be determined in part based on a type of surgery, one or more medical instruments used by a medical operator to perform the surgical procedure, an anatomical classification of a portion of the subject's body that is undergoing surgery (different steps or procedures may occur for different anatomies), and/or a similarity of a characteristic of the surgical procedure to another surgical procedure. In some cases, the one or more timing parameters may be determined in part based on a change in medical instruments used, a change in doctors or medical operators, a change in a position or an orientation of one or more medical instruments being used, a change in a position or an orientation of a doctor or a medical operator during a surgical procedure, and/or a change in a position or an orientation of a patient who is undergoing a surgical procedure.

In some cases, the one or more timing parameters may be generated based on an anatomy type of a patient. In such cases, a set of steps for a procedure for that anatomy type may be predicted, along with predicted timing for each step. An anatomy type of a patient may be recognized. In some embodiments, images from the plurality of videos may be used to recognize an anatomy type of the patient. In some instances, a patient's medical records may be automatically accessed and used to aid in recognition of the anatomy type of the patient. In some instances, medical personnel may input information that may be used to determine a patient's anatomy type. In some instances, the medical personnel may directly input the patient's anatomy type. In some instances, information from multiple sources (e.g., two or more of video images, medical records, manual input) may be used to determine the patient's anatomy type. Examples of factors that may affect a patient's anatomy type may include, but is not limited to, gender, age, weight, height, positioning of various anatomical features, size of various anatomical features, past medical procedures or history, presence or absence of scar tissue, or any other factors.

In some cases, the plurality of videos may be analyzed and used to aid in determining a patient's anatomy type. Object recognition may be utilized to recognize different anatomical features on a patient. In some instances, one or more feature points may be recognized and used to recognize one or more objects. In some embodiments, size and/or scaling may be determined between the different anatomical features. One or more fiducial markers may be provided on a patient to aid in determining scale and/or size.

In some embodiments, machine learning may be utilized in determining a patient's anatomy type. When the patient's information is provided and/or accessed, the systems and methods provided herein may automatically determine the patient's anatomy type. In some embodiments, the determined anatomy type may optionally be displayed to medical personnel. The medical personnel may be able to review the determined anatomy type and confirm whether the assessment is accurate. If the assessment is not accurate, the medical personnel may be able to correct the anatomy type or provide additional information that may update the anatomy type.

Since patients with different anatomical types may require different steps in order to achieve a similar goal, a prediction of a set of steps for a procedure and the associated timing for those predicted steps may depend on the anatomy type of a patient. Medical personnel may take different steps depending on a patient's placement or size of various anatomical features, age, past medical conditions, overall health, or other factors. In some instances, different steps may be taken for different anatomical types. For instances, certain steps or techniques may be better suited for particular anatomical features. In other instances, the same steps may be taken, but the timing may differ significantly. For instance, for a particular anatomical features, a particular step may be more difficult to perform, and may end up typically taking a longer time than if the anatomical feature was different.

In some embodiments, machine learning may be utilized in determining the steps to utilize for a particular anatomy type. The systems and methods provided herein may utilize training datasets in determining determine the steps that are typically used for a particular anatomy type. This may include determining timing of the various steps that are used. In some instances, the recommended steps may be displayed to the medical personnel. The steps may be displayed to the medical personnel before the medical personnel starts the procedure. The medical personnel may be able to review the recommended steps to confirm whether these recommendation is accurate. If the recommendation is not accurate or desirable, the medical personnel may provide some feedback or change the steps. The display may or may not include information about expected timing for the various steps.

In some cases, the one or more timing parameters may be used to generate or update an estimated or predicted timing of one or more steps of a surgical procedure. In some cases, the estimated timing of one or more steps of a surgical procedure may be updated based at least in part on an amount of progress associated with a surgical procedure.

The one or more timing parameters may be used to provide friends or family members of a medical patient with an estimate of how much of the surgical procedure is completed, how much time is remaining, and/or what steps are pending or completed. The friends or family members may be in a waiting room or another location that is remote from the location in which the surgical operation is being performed. In some cases, the one or more timing parameters may be used to provide a progress report for friends and family members in a waiting room. The progress report may comprise a % complete, a % remaining, a time left, and/or a time elapsed. In some cases, the progress report may notify or inform friends or family members when they can see the patient.

In some cases, the one or more timing parameters may be used to provide a progress report for other medical operators or medical personnel who may need to stay informed about the current progress of a surgical procedure. The other medical operators or medical personnel may be doctors or medical support staff who are performing another step in the surgical procedure. The other medical operators or medical personnel may be doctors or medical support staff who are performing a related or parallel procedure, such as in the case of donor and recipient surgical procedures. In some cases, the other medical operators or medical personnel may be doctors or medical support staff who are scheduled to operate in the same operating room in which the surgical procedure is being performed. The progress report may comprise a % complete, a % remaining, a time left, and/or a time elapsed. In some cases, the progress report may be used to prep other medical operators for timely tag-in, prep other medical instruments for use by the medical operator, prep medical personnel and support staff for room switching or patient room turnover, or provide an estimated timing for one or more steps of the surgical procedure to facilitate coordination of one or more steps of another parallel surgical procedure.

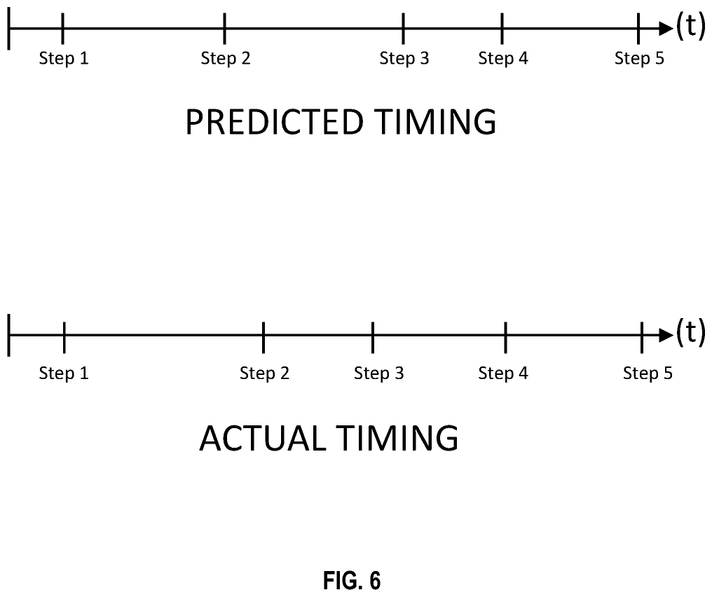

FIG. 6 illustrates a comparison of a predicted timing for one or more steps of a surgical procedure against an actual timing associated with the performance or completion of the one or more steps of the surgical procedure. In some instances, Step 1 of a surgical procedure may have a particular predicted timing, and Step 1 may actually be performed within approximately the same amount of time as predicted. This may cause no flags to be raised.

In another example, Step 2 of the surgical procedure may be expected to occur within a particular length of time, but in practice may actually take a significantly longer period of time. When a significant deviation occurs, this difference may be flagged, and the medical operator performing the surgical procedure may be notified. In some cases, when a signification deviation occurs between the predicted timing and the actual timing, other medical operators working on a parallel or concurrent procedure (e.g., in the case of donor and recipient surgeries) may be notified. In some cases, when a signification deviation occurs between the predicted timing and the actual timing, other medical personnel who are coordinating the scheduling of operating rooms for a health care facility may be notified.

In another example, Step 3 of the surgical procedure may be expected to occur within a particular length of time, but in practice may be completed before the predicted time. When a significant deviation occurs, this difference may be flagged, and the medical operator performing the surgical procedure may be notified. In some cases, when a signification deviation occurs between the predicted timing and the actual timing, other medical operators working on a parallel or concurrent procedure (e.g., in the case of donor and recipient surgeries) may be notified. In some cases, when a signification deviation occurs between the predicted timing and the actual timing, other medical personnel who are coordinating the scheduling of operating rooms for a health care facility may be notified.

In another example, Step 4 and Step 5 of the surgical procedure may be expected to occur within a particular length of time. Based on the actual timing of prior steps (e.g., Step 1, Step 2, and/or Step 3 of the surgical procedure), the predicted timing for Step 4 and Step 5 may be adjusted to better approximate the actual timing for Step 4 and Step 5 of the surgical procedure. In some cases, other medical operators working on a parallel or concurrent procedure (e.g., in the case of donor and recipient surgeries) may be notified of the updated predicted timing for subsequent steps of the surgical procedure. In some cases, other medical personnel who are coordinating the scheduling of operating rooms for a health care facility may be notified of the updated predicted timing for subsequent steps of the surgical procedure.

In any of the embodiments described herein, the predicted or estimated timing for one or more steps of a surgical procedure may be updated in real time based on a performance or completion of one or more steps of the surgical procedure. In any of the embodiments described herein, the updated predicted or estimated timing for one or more steps of a surgical procedure may be provided and/or transmitted to one or more end users in real time as one or more steps of the surgical procedure are being performed or completed. As used herein, the term “real time” may generally refer to a simultaneous or substantially simultaneous occurrence of a first event or action (e.g., performing or completing one or more steps of a surgical procedure) with respect to an occurrence of a second event or action (e.g., updating a predicted or estimated timing for one or more steps of a surgical procedure, or providing an updated predicted or estimated timing to one or more end users). A real-time action or event may be performed within a response time of less than one or more of the following: ten seconds, five seconds, one second, a tenth of a second, a hundredth of a second, a millisecond, or less relative to at least another event or action. A real-time action may be performed using one or more computer processors.

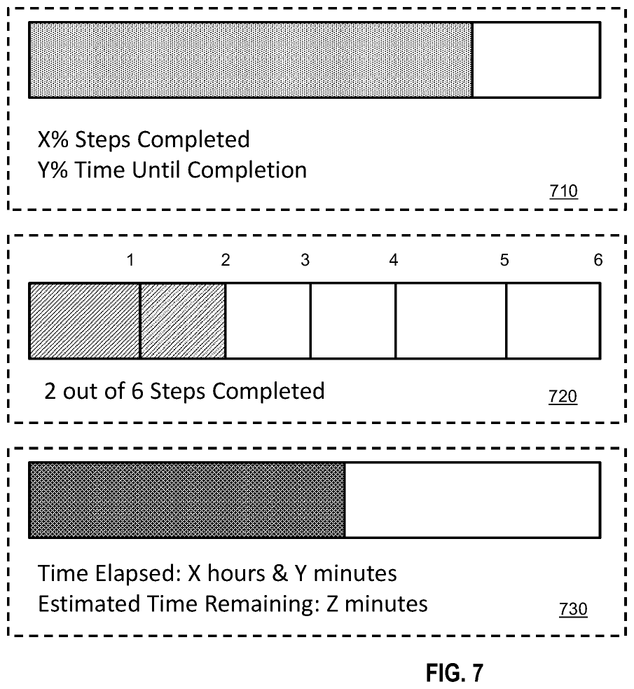

In some cases, the plurality of videos and/or the one or more timing parameters associated with the plurality of videos may be used to provide one or more status updates to one or more end users. The one or more status updates may be provided in real time or substantially in real time as one or more steps of a surgical procedure are being performed or completed. The one or more status updates may be provided in real time or substantially in real time as one or more videos are being captured by the plurality of imaging devices described herein. In some cases, the one or more status updates may comprise one or more status bars corresponding to a progress of a surgical procedure. The one or more end users may comprise other medical operators performing a parallel or concurrent procedure (e.g., in the context of donor and recipient surgical procedures), medical personnel helping to coordinate scheduling for operating rooms in a health care facility, or friends and family members of the medical patient undergoing a surgical procedure. As shown in FIG. 7, in one example, a first status bar 710 may be configured to show a percent completion. The percent completion may correspond to a number of steps completed in relation to a total number of steps, or an amount of time left to completion in relation to a total amount of time estimated to complete the surgical procedure. In another example, a second status bar 720 may be configured to show how many steps have been completed in relation to a total number of steps needed to complete a surgical procedure. In another example, a third status bar 730 may be configured to show an amount of time elapsed and/or an estimated time remaining to complete the surgical procedure. In some cases, different status bars may be presented to different end users depending on the type or identity of the end user. Alternatively, end users may select different status bars to view within a user interface displayed on an end user device.

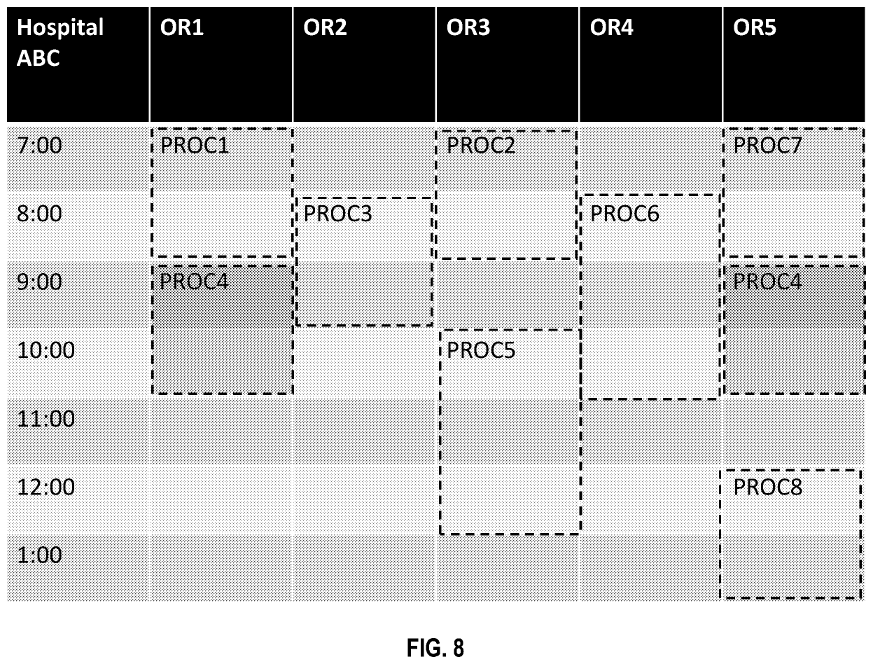

In some cases, the plurality of videos and/or the one or more timing parameters associated with the plurality of videos may be used to update scheduling information for a particular health care facility in real time. For example, as shown in FIG. 8, in Hospital ABC, there may be multiple locations where one or more surgical operations may be schedule. The multiple locations may include multiple operating rooms (e.g., OR1, OR2, OR3, OR 4, OR 5 etc.).

The scheduling information may include timing information, such as time of day for a particular day. In some instances, the scheduling information may be updated in real time. Updating scheduling information in real time may enable medical operators, practitioners, personnel, or support staff to anticipate changes in a timing associated with a performance or completion of one or more steps of a surgical procedure and to prepare for such changes accordingly. Such real time updates may provide medical operators, practitioners, personnel, or support staff with sufficient time to prepare operating rooms or medical tools and medical instruments for one or more surgical procedures. Such real time updates may also allow medical operators, practitioners, personnel, or support staff to coordinate the scheduling of a plurality of different surgical procedure within a health care facility and to manage the resources or staffing of the health care facility based on the latest timing information available. Scheduling information may be available for the current day, upcoming day, the next few days, the next week, the next month, etc. The scheduling information may be updated in real-time, or may be updated periodically (e.g., daily, every several hours, every hour, every 30 minutes, every 15 minutes, every 10 minutes, every 5 minutes, every minute, every second, or more frequently). The scheduling information may be updated in response to an event. The scheduling information may include information about a procedure that may occur at the various locations. The scheduling information may include information about when and where each scheduled surgical procedure at a health care facility will be performed for any given date.

In some cases, the scheduling information may include additional information about procedures. For example, Procedure 1 may be scheduled to occur at 7:00 AM in OR1. Procedure 4 may be scheduled to occur at 9:00 AM in OR 5. The procedures may be of different type. The estimated length of time for each procedure may or may not be provided. The estimated length of time for each procedure may be updated based on timing information derived from the plurality of videos captured by the plurality of imaging devices.

In some cases, if an actual timing for one or more steps of Procedure 1 is delayed or takes longer than a predicted timing for the one or more steps of Procedure 1 (i.e., if Procedure 1 takes longer than predicted or estimated), then an estimated completion time for Procedure 1 may be updated. Based on the updated estimated completion time for Procedure 1, Procedure 4 may be rescheduled for a different time or a different operating room at the same time. For example, Procedure 4 may be moved from OR 1 to OR 5.

In some cases, two or more surgical procedures may be coordinated in part based on the timing of one or more steps of the two or more surgical procedures. The two or more surgical procedures may comprise a first surgical procedure on a donor medical subject and a second surgical procedure on a recipient medical subject. At least a portion of the first surgical procedure and at least a portion of the second surgical procedure may be performed concurrently or simultaneously.

The systems and methods disclosed herein may be implemented to coordinate two or more surgical procedures to enable an optimal timing for the performance or completion of one or more steps in a first surgical procedure relative to the performance or completion of one or more steps in a second surgical procedure. Such optimal timing may help to reduce or minimize a time during which an organ being transferred from a donor to a recipient is outside of a body of the donor or the recipient. In some cases, if the performance or completion of one or more steps in a first surgical procedure is delayed, the systems and methods disclosed herein may be implemented to alert a medical operator performing a second surgical procedure to slow down. In some cases, if one or more steps in a first surgical procedure are being performed or completed ahead of schedule (i.e., faster than predicted or estimated), the systems and methods disclosed herein may be implemented to alert a medical operator performing a second surgical procedure to speed up.

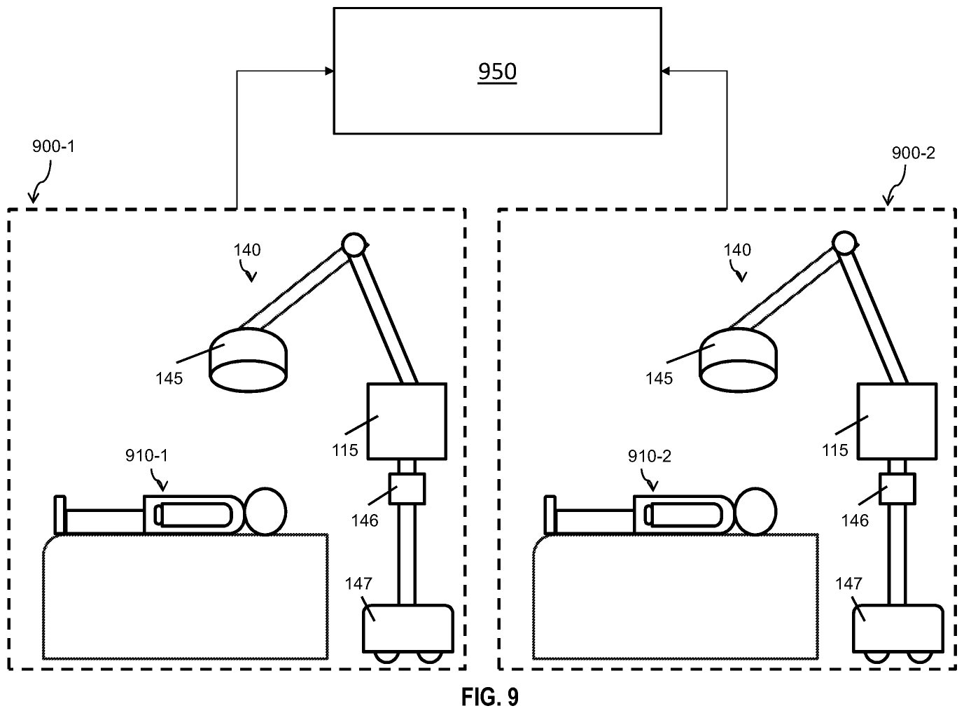

FIG. 9 illustrates a plurality of surgical procedures 900-1 and 900-2 for a donor medical subject and a recipient medical subject. In some cases, a first set of videos may be captured for a first surgical procedure on a donor 910-1. In some cases, a second set of videos may be captured for a second surgical procedure on a recipient 910-2. The first set of videos may be transmitted from a first location in which the first surgical procedure is being performed to a second location in which the second surgical procedure is being performed. The second set of videos may be transmitted from a second location in which the second surgical procedure is being performed to a first location in which the first surgical procedure is being performed. In some cases, the first set of videos and/or the second set of videos may be provided to a video processing module 950 in order to generate one or more timing parameters associated with the first surgical procedure and/or the second surgical procedure. In some cases, the first set of videos and/or the second set of videos may be provided to a video processing module 950 in order to update an estimated timing associated with one or more steps of the first surgical procedure and/or the second surgical procedure. In some cases, the first set of videos and/or the second set of videos may be provided to a video processing module 950 in order to generate one or more status bars associated with a progress of the first surgical procedure and/or the second surgical procedure. The plurality of videos, the one or more timing parameters associated with the first and/or second surgical procedures, the estimated timing associated with the first and/or second surgical procedures, or the status bars associated with the progress of the first and/or second surgical procedures may be provided to a first medical operator (i.e., a medical operator performing the first surgical procedure) or a second medical operator (i.e., a medical operator performing the second surgical procedure) in order to coordinate a performance or a completion of one or more steps of a donor or recipient surgical procedure.

In some cases, the plurality of videos captured by the plurality of imaging devices may be used to help one or more end users monitor a performance of one or more steps of the surgical procedure. For example, in some cases, one or more timing parameters derived from the plurality of videos may be provided to a medical operator or practitioner in real-time to inform the medical operator if he or she is on track, too slow, or ahead of schedule in relation to an estimated timeline associated with the surgical procedure. In some embodiments, the systems and methods provided herein may provide real-time support to the medical practitioner. While the medical practitioner is performing the procedure, helpful information for the procedure may be displayed and updated in real-time as steps are recognized. Any disparities from expected steps and/or timing may be noted to the medical practitioner.

In other cases, the one or more timing parameters may be provided to the medical operator after the surgical procedure is completed. In such cases, the medical operator may view and/or analyze his or her performance based on the plurality of videos and the one or more timing parameters associated with the plurality of videos. Further, a plurality of post-surgery analytical information derived from the plurality of videos may be provided to the medical operator so that the medical operator may assess which steps took more time than expected, which steps took less time than expected, and which steps took about as much time to complete as expected. The post-surgery analytical information may comprise one or more timing parameters associated with one or more steps of the surgical procedure. In some cases, the post-surgery analytical information may comprise information on which medical tools were used during which steps of the surgical procedure, information on a movement of the medical tools over time, and/or information on a movement of the surgical operator's hands during the surgical procedure. In some cases, the post-surgery analytical information may provide one or more tips to a medical operator on how to perform one or more steps of the surgical procedure in order to increase an efficiency of the medical operator during one or more steps of the surgical procedure.

In some cases, the plurality of videos captured by the plurality of imaging devices may be used for educational or training purposes. For example, the plurality of videos may be used to show medical students, interns, residents, or other doctors or physicians how to perform one or more steps of a surgical procedure. For instance, if a medical personnel is having difficulty with a particular step, the medical personnel may request a training video or a series of instructions to walk through the step. In some instances, if the medical personnel is having difficulty using a medical device or product, the medical personnel may request a training video or series of instructions to walk through use of the device or product. In some cases, the plurality of videos may be used to show medical students, interns, residents, or other doctors or physicians how not to perform one or more steps of a surgical procedure.

In some cases, the plurality of videos may be processed to provide one or more end users with video analytics data. The video analytics data may comprise information on a skill or an efficiency of a medical operator. In some cases, the video analytics data may provide an assessment of a level of skill or a level of efficiency of a medical operator in relation to other medical operators.

In some cases, the plurality of videos may be provided to an artificial intelligence recorder system. The artificial intelligence recorder system may be configured to analyze a performance of one or more steps of a surgical procedure by one or more medical operators.

In some embodiments, it may be desirable to assess medical personnel performance after a procedure has been completed. This may be useful as feedback to the medical personnel. This may allow the medical personnel to focus on improving in areas as needed. The medical personal may wish to know his or her own strengths and weaknesses. The medical personnel may wish to find ways to improve his or her own effectiveness and efficiency.

In some embodiments, it may be desirable for other individuals to assess medical personnel performance. For instance, a health care facility administrator, or a medical personnel's colleague or supervisor may wish to assess the performance of the medical personnel. In some embodiments, medical personnel performance assessment may be useful for assessing the individual medical personnel, or a particular group or department may be assessed as an aggregate of the individual members. Similarly, a health care facility or practice may be assessed as an aggregate of the individual members.

The artificial intelligence recorder system may be configured to assess medical personnel in any manner. In one example, the medical personnel may be given a score for a particular medical procedure. The score may be a numerical value, a letter grade, a qualitative assessment, a quantitative assessment, or any other type of measure of the medical personnel's performance. Any description herein of a score may apply to any other type of assessment.

In some cases, the practitioner's score may be based on one or more factors. For instance, timing may be provided as a factor in assessing practitioner performance. For instance, if the medical personnel is taking much longer than expected to perform medical procedures, or certain steps of medical procedures, this may reflect detrimentally on the medical personnel's assessment. If the medical personnel has a large or significant deviation from expected time to completion for a medical procedure, this may detrimentally affect his or her score. Similarly, if the medical personnel takes less time than expected to perform the medical procedure, or certain steps of medical procedure, which may positively affect his or her assessment. In some instances, threshold values may be provided before the deviation is significant enough to affect his or her score positively or negatively. In some instances, the greater the deviation, the more that the timing affects his or her score. For example, if a medical personnel's time to complete a procedure is 30 minutes over the expected time, this may impact his score more negatively than if the medical personnel's time to complete the procedure is 10 minutes over the expected time. Similarly, if the medical personnel completes a procedure 30 minutes early, this may impact his score more positively than if the medical personnel's time to complete the procedure is 5 minutes under the expected time.

Other factors may be used to assess medical personnel performance. For instance, the effectiveness or outcome of the procedure may be a factor that affects the medical personnel's assessment. If complications arise, or if the medical personnel makes a mistake, this may negatively affect the medical personnel's score. Similarly, if the medical personnel has a complication-free procedure, this may positively affect the medical personnel's score. In some instances, recovery of the patient may be taken into account when assessing the performance of the medical personnel.

Another factor that may be taken into account is cost. For example, if the medical personnel uses more medical products or devices than expected, then this may add to the cost, and may negatively affect the medical personnel's assessment. For instance, if the medical personnel regularly drops objects, this may reflect detrimentally on the medical personnel's assessment. Similarly, if the medical personnel uses more resources (e.g., devices, products, medication, instruments, etc.) than expected, the cost may go up. Similarly, if the procedure takes longer than expected, the corresponding costs may also go up.

In some cases, the artificial intelligence recorder system may be configured to use the plurality of videos and/or medical practitioner scores to create a model of one or more exemplary ways to perform a surgical procedure. The model may provide end users (e.g., a medical operator, a medical student, an intern, or a resident) with a visualization and/or a description for how to perform one or more steps of a surgical procedure. The model may be configured to provide different users with different methods for performing one or more steps of a surgical procedure based on a skill or a level of experience of an operator. The model may be configured to provide users with different methods for performing one or more steps of a surgical procedure based on a current step of the surgical procedure or the current status or condition of the patient.

In some cases, the artificial intelligence recorder system may be configured to provide end users with a visualization of a model way to perform a surgery and/or a model way to execute one or more steps of a surgical procedure. In such cases, the artificial intelligence recorder system may be configured to provide end users with at least a subset of the plurality of videos captured the one or more imaging devices. The plurality of videos may have additional data, annotations, descriptions, or audio overlaid on top of the plurality of videos for educational or training purposes. In some cases, the plurality of videos may be provided to end users through live streaming over a communications network. In some cases, the plurality of videos may be accessed by through a video broadcast channel after the surgical procedure is completed. In some cases, the plurality of videos may be provided through a video on demand system, whereby end users may search for or look up model ways on how to perform one or more steps of a surgical procedure. The artificial intelligence recorder system may also provide post-procedure analysis and feedback. In some embodiments, a score for a practitioner's performance may be generated. The practitioner may be provided with an option to review the video, and the most relevant portions may be automatically recognized and brought to the front so that the practitioner does not need to spend extra time sorting or searching through irrelevant videos.

In some cases, the artificial intelligence recorder system may be configured to anonymize data that may be associated with one or more patients. For example, the artificial intelligence recorder system may be configured to redact, block, or screen information displayed on the plurality of videos that are provided to end users for educational or training purposes.

In some cases, the artificial intelligence recorder system may be configured to provide smart translations. The smart translations may build therapy-specific language models that may be used to buttress various language translation with domain specific language. For instance, for particular types of procedures or medical areas, various vernacular may be used. Different medical personnel may use different terms for the same meaning. The systems and methods provided herein may be able to recognize the different terms used and normalize the language.

Smart translations may apply to commands spoken by medical personnel during a medical procedure. The medical personnel may ask for support or provide other verbal commands. The medical console or other devices may use the smart translations. This may help the medical console and other devices recognize commands provided by the medical personnel, even if the language is not standard.

In some instances, a transcript of the procedure may be formed. One or microphones, such as an audio enhancement module, may be used to collect audio. One or more members of the medical team may speak during the procedure. In some instances, this may include language that relates to the procedure. The smart translations may automatically include translations of terminology used in order to conform to the medical practice. For instance, for certain procedures, certain standard terms may be used. Even if the medical personnel use different terms, the transcript may reference the standard terminology. In some embodiments, the transcript may include both the original language as well as the translations.

In some instances, when individuals are speaking with one another via one or more communication devices, the smart translations may automatically offer up the standard terminology as needed. If one user is speaking or typing to another user and utilizing non-standard terminology, the smart translations may automatically conform the language to standard terminology. In some instances, each medical area or specialty may have its own set of standard terminology. Standard terminology may be provided within the context of a procedure being conducted.

Optionally, the systems and methods provided herein may support multiple languages. For example, an operating room may be located within the United States with the medical personnel speaking English. An individual providing remote support may be located in Germany and may speak German. The systems and methods provided herein may translate between different languages. The smart translations may be employed so that the standard terminology is used in each language. Even if different words or phrasing is used by the individuals, the smart technology may make sure the words that are translated conform to the standard terminology in each language with respect to the medical procedure.

The smart translations may be supported locally at a medical console. The smart translations may occur on-board the medical console. Alternatively, the smart translations may occur at one or more remote servers. The smart translations may be implemented through a cloud computing infrastructure. For instance, the smart translations may occur in the cloud and be pushed back to the relevant consoles.

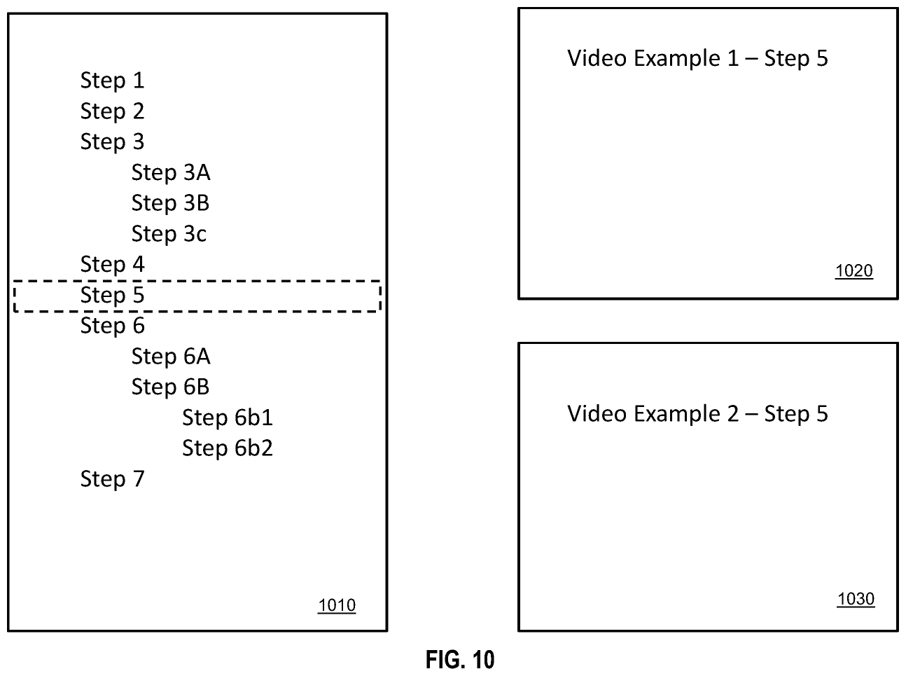

FIG. 10 illustrates an example of one or more user interfaces that may be generated by an artificial intelligence recorder system so that end users may view different model ways to perform one or more steps of a surgical procedure. In some instances, the artificial intelligence recorder system may be configured to analyze one or more videos of a surgical procedure and generate an interactive user interface 1010 that allows end users to view a list of steps associated with the surgical procedure. In some cases, an end user may use the artificial intelligence recorder system to search for a particular type of surgical procedure or a particular model way to perform one or more steps of a surgical procedure. In such cases, the interactive user interface 1010 may be configured to generate or update the list of steps displayed to the end user based on a particular type of surgical procedure selected by the end user.

The interactive user interface 1010 may be configured to allow an end user to select one or more steps of a surgical procedure in order to view one or more model ways to perform the one or more selected steps of the surgical procedure. For example, an end user may use the interactive user interface 1010 to select Step 5. When the end user selects Step 5, one or more videos 1020 and 1030 may be displayed for the end user. A first video 1020 may show the end user a first exemplary way to perform Step 5 of a particular surgical procedure. A second video 1030 may show the end user a second exemplary way to perform Step 5 of a particular surgical procedure. The one or more videos 1020 and 1030 may comprise at least a portion of the plurality of videos captured using the plurality of imaging devices described herein.

In some cases, the plurality of videos captured by the plurality of imaging devices may be distributed to one or more end users using a broadcasting system. The broadcasting system may be configured to distribute at least a subset of the plurality of videos to one or more end user devices (e.g., a mobile device, a smartphone, a tablet, a desktop, a laptop, or a television) for viewing. The broadcasting system may be configured to connect to one or more end user devices using any one or more communication networks as described herein. The broadcasting system may be configured to transmit at least a subset of the plurality of videos to one or more end users via one or more channels. The one or more end users may connect to and/or tune into the one or more channels to view one or more videos of one or more surgical procedures being performed in real time. In some cases, one or more end users may connect to and/or tune into the one or more channels to view one or more saved videos of one or more surgical procedures that were previously performed and/or completed.

In some cases, the broadcasting system may be configured to allow one or more end users to select one or more videos for viewing. The one or more videos may correspond to different surgical procedures. The one or more videos may correspond to various steps of a surgical procedure. The one or more videos may correspond to one or more examples or suggested methods of how to perform one or more steps of a surgical procedure. The one or more videos may correspond to one or more model ways to perform a surgical procedure. In some cases, the one or more videos may correspond to a performance of a particular surgical procedure by one or more medical practitioners. In some cases, the one or more videos may correspond to a performance of a particular surgical procedure by a particular medical practitioner.

In some cases, the broadcasting system may be configured to allow one or more end users to search for one or more videos for viewing. For example, the one or more end users may search for one or more videos based on a type of surgical procedure, a particular step of a surgical procedure, or a particular medical operator who is experienced in performing one or more steps of a surgical procedure. In some cases, the one or more end users may search for one or more videos based on a score or an efficiency of a medical operator who is performing or has performed a surgical procedure. In another example, the one or more end users may search for one or more videos by browsing through one or more predetermined categories for different types of surgical procedures. In another example, the one or more end users may search for one or more videos based on whether the one or more videos are live streams of a surgical procedure being performed live or saved videos of a surgical procedure that has already been performed or completed. In some cases, the broadcasting system may be configured to suggest one or more videos based on the type of end user, the identity of the end user, and/or a search history or viewing history associated with the end user.

As described above, the one or more videos available for searching and/or viewing using the broadcasting system may have one or more redacted portions to cover, block, or remove personal information associated with a medical patient or subject who is undergoing a surgical procedure within the one or more videos. In some cases, the one or more videos available for searching and/or viewing using the broadcasting system may be augmented with smart translations as described above. In other cases, the one or more videos available for searching and/or viewing using the broadcasting system may be augmented with additional information such as annotations, commentary by one or more medical practitioners, and/or supplemental data from an EKG/ECG or one or more sensors for monitoring a heart rate, a blood pressure, an oxygen saturation, a respiration, and/or a temperature of the subject undergoing the surgical procedure.

Video Collaboration

In some embodiments, the video collaboration systems of the present disclosure may be adapted, configured, and/or implemented to enable sharing of media content (e.g., videos) between remote users (e.g., a product or medical device specialist) and medical personnel in a healthcare facility. In some cases, the video collaboration systems of the present disclosure may be adapted, configured, and/or implemented to facilitate the transmission and sharing of media content from a product or medical device specialist to a doctor or a surgeon who is preparing for a surgical procedure or who is performing one or more steps in a surgical procedure. In any of the embodiments described herein, the media content may comprise images, videos, and/or medical data pertaining to a surgical procedure or a medical device that is usable to perform one or more steps of the surgical procedure.

In some cases, a virtual workspace may be provided for one or more remote end users (e.g., a product or medical device specialist) to manage, organize, and/or stage media content so that the media content can be displayed, presented, and/or shared with medical personnel in a healthcare facility. The media content may comprise images, videos, and/or medical data corresponding to an operation or a usage of a medical device or instrument. In some cases, the media content may comprise images, videos, and/or medical data that can be used to instruct, guide, and/or train one or more end users to perform one or more steps in a surgical procedure.

In some embodiments, the media content may comprise product demo materials and/or videos from a company-specific video library. The company-specific video library may correspond to a library or collection of images and/or videos that is created and/or managed by a medical device manufacturer or a medical device supplier. The company-specific video library may correspond to a library or collection of images and/or videos that is created and/or managed by one or more product specialists working for a medical device company (e.g., a medical device manufacturer or a medical device supplier). The media content within the company-specific video library may be used to instruct, guide, and/or train one or more end users on how to use a medical device, instrument, or tool during a surgical procedure.

In some embodiments, the media content may comprise pre-procedural video clips or images. The pre-procedural video clips or images may be of a specific patient (e.g., the patient that will be undergoing a surgical procedure under the direction or supervision of a medical worker who has access to the media content). In such cases, the systems of the present disclosure may be integrated into the electronic records systems or the picture archiving and communication systems of a healthcare facility. In some embodiments, the media content may comprise non-patient specific sample case images or videos to help local doctors better understand or follow the guidance, training, instructions, or remote consultations provided by a remote user (e.g., a medical device specialist).

In some embodiments, the media content may comprise images and/or video clips from a live or ongoing procedure. In some cases, the media content may be locally stored by a remote user (e.g., a remote product specialist) for use during a surgical procedure. In such cases, the media content may be deleted after the surgical procedure is completed, after one or more steps of the surgical procedure are completed, or after a predetermined amount of time. In some cases, the virtual workspace may be configured to provide a remote user the ability to record one or more videos that are temporarily stored on a cloud server, in order to comply with HIPPA. The one or more videos may be limited to a predetermined length (e.g., less than a minute, less than 30 seconds, less than 20 seconds, less than 10 seconds, etc.). The one or more videos may be pulled back into the procedure and presented to a surgical operator or medical worker as needed while the surgical operator or medical worker is performing one or more steps of a surgical procedure, or preparing to execute one or more steps of a surgical procedure.

In some cases, a remote user (e.g., a medical device representative) may create or compile an anonymized video library comprising one or more anonymized images and/or videos captured during a medical procedure. The one or more anonymized images and/or videos may be edited or redacted to conceal or remove a medical subject's personal information. These images and/or videos may be stored in a cloud server under the remote user's personal account. The medical device representative may be a specialist with respect to a medical procedure or a medical device that is usable to perform one or more steps of the medical procedure. In some cases, the medical device representative may be permitted to share the anonymized images and/or videos with a doctor or a surgeon during a surgery procedure.

In some embodiments, the virtual workspace may be configured to allow a remote representative to utilize subscription video on demand (SVOD), transactional video on demand (TVOD), premium video on demand (PVOD), and/or advertising video on demand (AVOD) services. Once the remote representative has purchased and/or subscribed to certain media content available through the SVOD, TVOD, PVOD, and/or AVOD services, the virtual workspace may permit the remote representative to provide the media content to a doctor or a surgeon who is performing a surgical procedure or who is preparing to perform one or more steps of a surgical procedure.

In some cases, one or more videos of a medical or surgical procedure may be obtained using a plurality of cameras and/or imaging sensors. The systems and methods of the present disclosure may provide the ability for one or more users (e.g., surgeon, medical worker, assistant, vendor representative, remote specialist, medical researcher, or any other individual interested in viewing and providing inputs, thoughts, or opinions on the content of the one or more videos) to join a virtual session (e.g., a virtual video collaboration conference) to create, share, and view annotations to the one or more videos. The virtual session may permit one or more users to view the one or more videos of the medical or surgical procedure live (i.e., in real time) as the one or more videos are being captured. Alternatively, the virtual session may permit one or more users to view medical or surgical videos that have been saved to a video library after the performance or completion of one or more steps in a surgical procedure.

The virtual session may provide the one or more users with a user interface that permits the users to provide the one or more annotations or markings to the one or more videos. The annotations may comprise, for example, a text-based annotation, a visual annotation (e.g., one or more lines or shapes of various sizes, shapes, colors, formatting, etc.), an audio-based annotation (e.g., audio commentary relating to a portion of the one or more videos), or a video-based annotation (e.g., audiovisual commentary relating to a portion of the one or more videos).

In some cases, the one or more annotations may be manually created or provided by the user as the user reviews the one or more videos. In other cases, the user may select one or more annotations from a library of annotations and manually place or position the annotations onto a portion of the one or more videos. In some cases, the one or more annotations may comprise, for example, a bounding box that is generated or placed around one or more portions of the videos. In some cases, the one or more annotations may comprise a zero-dimensional feature that is generated within the one or more videos. In some instances, the zero-dimensional feature may comprise a dot. In some cases, the one or more annotations may comprise a one-dimensional feature that is generated within the one or more videos. In some instances, the one-dimensional feature may comprise a line, a line segment, or a broken line comprising two or more line segments. In some cases, the one-dimensional feature may comprise a linear portion. In some cases, the one-dimensional feature may comprise a curved portion. In some cases, the one or more annotations may comprise a two-dimensional feature that is generated within the one or more videos. In some cases, the two-dimensional feature may comprise a circle, an ellipse, or a polygon with three or more sides. Alternatively, the two-dimensional feature may comprise any amorphous, irregular, indefinite, random, or arbitrary shape. Such amorphous, irregular, indefinite, random, or arbitrary shape may be drawn or generated by the user using one or more input devices (e.g., a computer mouse, a laptop trackpad, or a mobile device touch screen). In some cases, two or more sides of the polygon may comprise a same length. In other cases, two or more sides of the polygon may comprise different lengths. In some cases, the two-dimensional feature may comprise a shape with two or more sides having different lengths or different curvatures. In some cases, the two-dimensional feature may comprise a shape with one or more linear portions and/or one or more curved portions. In some cases, the two-dimensional feature may comprise an amorphous shape that does not correspond to a circle, an ellipse, or a polygon. In some cases, the two-dimensional feature may comprise an arbitrary shape that is drawn or generated by an annotator (e.g., the user reviewing the one or more videos).

In some cases, the annotations may comprise, for example, a predetermined shape (e.g., a circle or a square) that may be placed or overlaid on the one or more videos. The predetermined shape may be positioned or repositioned using a click to place or drag and drop operation. In other cases, the annotations may comprise, for example, any manually drawn shape generated by the user using an input device such as a computer mouse, a mobile device touchscreen, or a laptop touchpad. The manually drawn shape may comprise any amorphous, irregular, indefinite, random, or arbitrary shape. In some alternative embodiments, the annotations may comprise an arrow or a text-based annotation that is placed on or near one or more features or regions appearing in the one or more videos.

The virtual session may permit multiple users to make live annotations simultaneously. In some cases, the virtual session may permit users to make and/or share live annotations only during specific time periods assigned or designated for each user. For example, a first user may only make and/or share annotations during a first part of a surgical procedure, and a second user may only make and/or share annotations during a second part of the surgical procedure. Sharing the annotations may comprise broadcasting or rebroadcasting the one or more videos with the user-provided annotations to other users in the virtual session. Broadcasting of such videos containing the user-provided annotations may occur substantially simultaneously with the broadcasting of the original videos to the users within the virtual session. This may allow the annotations to be streamed live to the other users in the virtual session as the one or more videos are being streamed to and viewed by the various users in the virtual session, without interruption of the viewing experience. Rebroadcasting may comprise broadcasting the videos containing the user-provided annotations at a later time after the broadcasting of the original videos to the users within the virtual session.

In some embodiments, the virtual session may permit users to provide additional annotations on top of or in addition to the annotations provided by another user. In some cases, each user may provide his or her own annotations in parallel and share the annotations live with the other users. The other users may then provide additional annotations for sharing or broadcasting to the users in the virtual session.

In some cases, the virtual session may permit the users to modify, adjust, or change the content of the one or more videos, in addition to providing one or more annotations. Such modifications, adjustments, or changes may comprise, for example, adding or removing audio and/or visual effects using one or more post-processing steps. In some cases, the modifications, adjustments, or changes may comprise adding additional data (e.g., data obtained using one or more sensors and/or medical tools or instruments) to the one or more videos. The virtual session may be configured to permit a user to broadcast and/or rebroadcast the one or more videos containing modifications, adjustments, or changes to the content of the videos with various other users in the virtual session. In some cases, the virtual session may permit broadcasting and/or rebroadcasting to all of the users in the virtual session. In other cases, the virtual session may permit broadcasting and/or rebroadcasting to a particular subset of the users in the virtual session. The subset of the users may be determined based on medical specialty, or may be based on a manual input or selection of a desired subset of users.

In some cases, one or more videos of a medical or surgical procedure may be obtained using a plurality of cameras and/or imaging sensors. The one or more videos may be saved to a local storage device (e.g., a storage drive of a computing device). Alternatively, the one or more videos may be uploaded to and/or saved on a server (e.g., a remote server or a cloud server). The one or more videos (or a particular subset thereof) may be pulled from the storage device or server for access and viewing by a user. The particular videos pulled for access and viewing may be associated with a particular view of a surgical procedure, or a particular camera and/or imaging sensor used during the surgical procedure. The one or more videos saved to the local storage device or the server may be streamed or broadcasted to a plurality of users via the virtual sessions described elsewhere herein.

In some embodiments, a plurality of remote users may join the virtual session or workspace to collectively view one or more videos of a surgical procedure, and to collaborate with one another based on the one or more videos. Such collaboration may involve, for example, a first remote specialist recording a portion of the one or more videos, telestrating on top of the recorded portion of the one or more videos, and streaming or broadcasting the recorded portion containing the one or more telestrations to a second remote specialist or at least one other individual. The at least one other individual may be, for example, someone who is either (a) remote from the healthcare facility in which the surgical procedure is being conducted, or (b) in or near the healthcare facility in which the surgical procedure is being conducted. As used herein, telestrating may refer to providing one or more annotations or markings to an image, a video, or a recording of a video that was previously streamed or that is currently being streamed live. As used herein, telestration may refer to one or more annotations or markings that can be provided or overlaid on an image, a video, or a recording of a video (e.g., using a finger, a stylus, a pen, a touchscreen, a computer display, or a tablet display). The telestrations may be provided based on a physical input, or based on an optical detection of one or more movements or gestures by the user providing the telestrations.

In some cases, while one or more videos of a live surgical procedure are being streamed, multiple specialists can join in on the virtual session to record various portions of the ongoing surgical procedure, telestrate on the recordings respectively captured by each specialist, and simultaneously stream back the recordings containing the telestrations to (i) the other specialists in the virtual session, or (ii) an individual who is in or near the healthcare facility in which the surgical procedure is being performed (e.g., the doctor or surgeon performing the surgical procedure). Such simultaneous streaming and sharing of the recordings containing the telestrations can allow the various remote specialists to compare and contrast their interpretations and evaluations of the surgical procedure, including whether or not a step is being performed correctly, and if the surgeon performing the procedure can make any adjustments or improvements to increase efficiency or minimize risk.

In some cases, the virtual session may permit the multiple specialists to simultaneously share their screens. In such instances, a first specialist can show a second specialist live telestrations that the first specialist is providing on the one or more videos while the second specialist also shows another specialist (e.g., the first specialist and/or another third specialist) telestrations that the second specialist is providing on the one or more videos. In some cases, the virtual session may permit the multiple specialists to simultaneously share individual recordings of the one or more videos. Such one or more individual recordings may correspond to different portions of the one or more videos, and may be of different lengths. Such individual recordings may be pulled from different cameras or imaging sensors used to capture the one or more videos of the surgical procedure. Such individual recordings may or may not comprises one or more telestrations, annotations, or markings provided by the specialist who initiated or captured the recording. For example, a first specialist may share a first recording corresponding to a first portion of the one or more videos, and a second specialist may share a second recording corresponding to a second portion of the one or more videos. The first portion and the second portion of the one or more videos may be selected by the specialist based on his or her interest or expertise in a particular stage or step of the surgical procedure. During such simultaneous sharing of individual recordings, a first specialist can show a second specialist live telestrations that the first specialist is providing on the one or more recorded videos while the second specialist also shows another specialist (e.g., the first specialist and/or another third specialist) telestrations that the second specialist is providing on the one or more recorded videos. Such simultaneous sharing of recordings and telestrations can allow the specialists to compare and contrast the benefits, advantages, and/or disadvantages of performing a surgical procedure in various different ways or fashions.

In some instances, simultaneous streaming and sharing of video recordings and live telestrations can allow a first remote specialist to see telestrations provided by a second and third remote specialist at the same time. In some cases, the second remote specialist can provide a first set of telestrations corresponding to a first method of performing a surgical procedure, and the third remote specialist can provide a second set of telestrations corresponding to a second method of performing the surgical procedure. The first remote specialist can view both the first and the second set of telestrations to compare the first and second methods of performing the surgical procedure. The first remote specialist can use both the first and the second set of telestrations to evaluate improvements that can be obtained (e.g., in terms of surgical outcome, patient safety, or operator efficiency) if the surgical procedure is performed in accordance with the various methods suggested or outlined by the telestrations provided by each remote specialist.

In some instances, simultaneous streaming and sharing of video recordings and live telestrations can allow a first user (e.g., a doctor or a surgeon performing a surgical procedure) to see telestrations provided by a second and third user at the same time. In some cases, the second user can provide a first set of telestrations corresponding to a first method of performing a surgical procedure, and the third user can provide a second set of telestrations corresponding to a second method of performing the surgical procedure. The first user can view both the first and the second set of telestrations to compare the first and second methods of performing the surgical procedure. The first user can use both the first and the second set of telestrations to evaluate improvements that can be obtained (e.g., in terms of surgical outcome, patient safety, or operator efficiency) if the surgical procedure is performed in accordance with the various methods suggested or outlined by the telestrations provided by each of the other users. The second user and the third user may be, for example, remote specialists who can provide feedback, commentary, guidance, or additional information to assist the first user while the first user is performing the surgical procedure, to provide additional training to the first user after the first user completes one or more steps of the surgical procedure, or to evaluate the first user's performance after completion of one or more steps of the surgical procedure.

In some instances, a first user (e.g., a first doctor or surgeon or medical specialist) can provide and share telestrations to show how a procedure should be completed. In some cases, a second user (e.g., a second doctor or surgeon or medical specialist) can provide separate telestrations (e.g., telestrations provided on a separate recording or a separate stream/broadcasting channel) to allow a third user (e.g., a third doctor or surgeon or medical specialist) to compare and contrast the various telestrations. In other cases, a second user (e.g., a second doctor or surgeon or medical specialist) can provide telestrations on top of the first user's telestrations to allow a third user (e.g., a third doctor or surgeon or medical specialist) to compare and contrast the various telestrations in a single recording, stream, or broadcast.

In some embodiments, the user or remote specialist who is sharing content (e.g., video recordings or telestrations) with the other users or specialists can share such content as a downloaded or downloadable file, or by providing access to such content via a server. Such server may be, for example, a cloud server.

In some cases, multiple users can telestrate the videos at the same time, and change the content of the videos by adding additional data or by changing some of the data associated with the videos (e.g., removing audio or post-processing the video). After the multiple users add additional data to the videos and/or change some of the data associated with the videos, the multiple users can re-broadcast the video containing the changed or modified content to other users (e.g., other remote specialists, or other individuals assisting with the surgical procedure). In some cases, the multiple users can provide further annotations or telestrations on top of the rebroadcasted videos containing various telestrations provided by other users, and to share such additional annotations or telestrations with the other users. In some cases, each of the users in the virtual session may provide their own telestrations in parallel and simultaneously share the telestrations such that each user sees multiple telestrations from other users corresponding to (i) the same portion or recording of a surgical video, (ii) various different portions or recordings of a surgical video or (iii) different views of the same portion or recording of a surgical video. Multiple users can telestrate at the same time and/or modify the telestrations that are provided by the various users at the same time. The telestrations may be provided on a live video stream of a surgical procedure or a recording (e.g., a video recording) of the surgical procedure. The multiple simultaneous telestrations by the multiple users may be provided with respect to the same live video stream or the same recording, in which case the multiple telestrations may be provided on top of one another. Alternatively, the multiple simultaneous telestrations by the multiple users may be provided with respect to different videos or recordings.

In some cases, the telestrations may be provided on a highlight video corresponding to various portions or sections of interest within a surgical video or a recording thereof. For example, a first user may provide a first set of telestrations associated with one or more portions or sections of interest within a surgical video. The telestrations may be shared, streamed, or broadcasted to other users. In some cases, multiple users may provide multiple sets of telestrations (e.g., separate telestrations on separate recordings, or a plurality of telestrations overlaid on top of each other). Such multiple sets of telestrations may be simultaneously streamed to and viewable by various users in the virtual session to compare and contrast various methods and guidance suggested or outlined by the various telestrations provided by the multiple users. In some cases, such multiple sets of telestrations may be simultaneously streamed to and viewable by various users in the virtual session to evaluate different ways to perform one or more steps of the surgical procedure to obtain different results (e.g., different surgical outcomes, or differences in operator efficiency or risk mitigation). In some cases, such multiple sets of telestrations may be simultaneously streamed to and viewable by various users in the virtual session so that the various users can see one or more improvements that can result from performing the surgical procedure in different ways according to the different telestrations provided by different users.

In some embodiments, the telestrations may be provided at a first time point of interest and a second time point of interest. The first time point of interest and/or the second time point of interest may correspond to one or more critical steps in the surgical procedure. The multiple users may provide multiple telestrations at the first time point of interest and/or the second time point of interest. The users may view the multiple telestrations simultaneously to see how outcomes or results at the second time point of interest change based on different actions taken at the first time point of interest. In some cases, the multiple telestrations may be provided with respect to different highlight videos so that a single user can see which steps or time points of a surgical procedure can impact a surgical outcome, and compare or contrast the various methods for performing such steps during such time points to improve the surgical outcome. As used herein, surgical outcome may correspond to an end result of a surgical procedure, a level of success of the surgical procedure, a level of risk associated with the performance of the surgical outcome, or an efficiency of the operator performing the surgical procedure.

In some embodiments, when a user (e.g., a specialist) telestrates on top of one or more videos or recordings, the user can share the one or more videos with other users (e.g., other specialists) at the same time. Further, the user may share multiple applications or windows at the same time along with the one or more videos or recordings having the telestrations provided by that user. This allows other users or specialists to view (i) the one or more videos or recordings having the telestrations and (ii) one or more applications or windows comprising additional information or content associated with the surgical procedure, in parallel or simultaneously. Such additional information or content may comprise, for example, medical or surgical data, reference materials pertaining to a performance of the surgical procedure or a usage of one or more tools, or additional annotations or telestrations provided on various videos or recordings of the surgical procedure. Allowing users or specialists to share one or more videos, applications, and/or windows at the same time with other users or specialists permits the other users or specialists to view, interpret, and analyze the shared videos or recordings containing one or more telestrations with reference to additional information or content. Such additional information or content can provide additional background or context for understanding, interpreting, and analyzing the shared videos or recordings and/or the telestrations provided on the shared videos or recordings.

Computer Systems

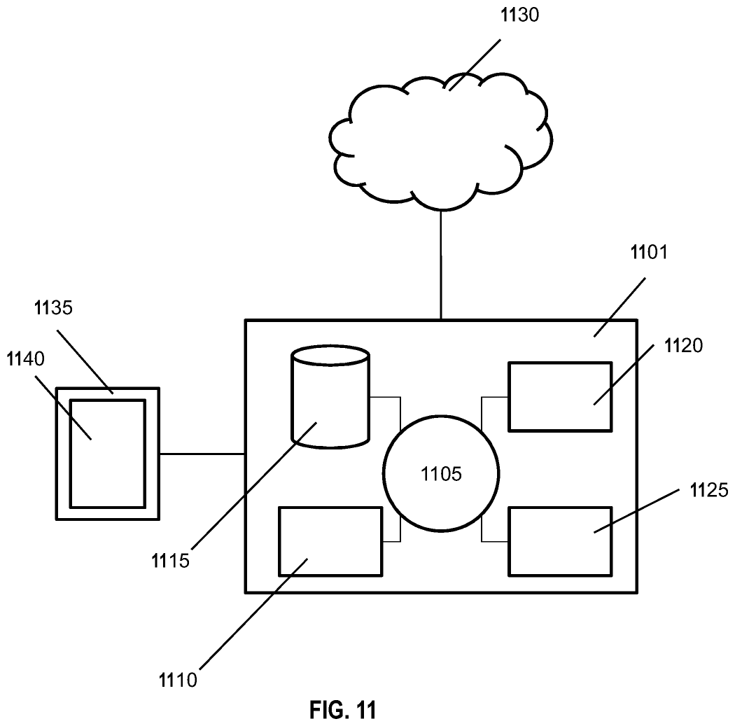

Another aspect of the present disclosure provides computer systems that are programmed or otherwise configured to implement methods of the disclosure. FIG. 11 shows a computer system 1101 that is programmed or otherwise configured to implement a method for video collaboration. The computer system 1101 may be configured to (a) obtain a plurality of videos of a surgical procedure; (b) determine an amount of progress for the surgical procedure based at least in part on the plurality of videos; and (c) update an estimated timing of one or more steps of the surgical procedure based at least in part on the amount of progress. The computer system 1101 may be further configured to provide the estimating timing to one or more end users to coordinate another surgical procedure or patient room turnover. In some cases, the computer system 1101 may be configured to (a) obtain a plurality of videos of a surgical procedure, wherein the plurality of videos are captured using a plurality of imaging devices; and (b) provide the plurality of videos to a plurality of end users, wherein each end user of the plurality of end users receives a different subset of the plurality of videos. The computer system 1101 can be an electronic device of a user or a computer system that is remotely located with respect to the electronic device. The electronic device can be a mobile electronic device.

The computer system 1101 may include a central processing unit (CPU, also “processor” and “computer processor” herein) 1105, which can be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120 and peripheral devices 1125 are in communication with the CPU 1105 through a communication bus (solid lines), such as a motherboard. The storage unit 1115 can be a data storage unit (or data repository) for storing data. The computer system 1101 can be operatively coupled to a computer network (“network”) 1130 with the aid of the communication interface 1120. The network 1130 can be the Internet, an internet and/or extranet, or an intranet and/or extranet that is in communication with the Internet. The network 1130 in some cases is a telecommunication and/or data network. The network 1130 can include one or more computer servers, which can enable distributed computing, such as cloud computing. The network 1130, in some cases with the aid of the computer system 1101, can implement a peer-to-peer network, which may enable devices coupled to the computer system 1101 to behave as a client or a server.

The CPU 1105 can execute a sequence of machine-readable instructions, which can be embodied in a program or software. The instructions may be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and writeback.

The CPU 1105 can be part of a circuit, such as an integrated circuit. One or more other components of the system 1101 can be included in the circuit. In some cases, the circuit is an application specific integrated circuit (ASIC).

The storage unit 1115 can store files, such as drivers, libraries and saved programs. The storage unit 1115 can store user data, e.g., user preferences and user programs. The computer system 1101 in some cases can include one or more additional data storage units that are external to the computer system 1101, such as located on a remote server that is in communication with the computer system 1101 through an intranet or the Internet.

The computer system 1101 can communicate with one or more remote computer systems through the network 1130. For instance, the computer system 1101 can communicate with a remote computer system of a user (e.g., an end user, a medical operator, medical support staff, medical personnel, friends or family members of a medical patient undergoing a surgical procedure, etc.). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1101 via the network 1130.

Methods as described herein can be implemented by way of machine (e.g., computer processor) executable code stored on an electronic storage location of the computer system 1101, such as, for example, on the memory 1110 or electronic storage unit 1115. The machine executable or machine readable code can be provided in the form of software. During use, the code can be executed by the processor 1105. In some cases, the code can be retrieved from the storage unit 1115 and stored on the memory 1110 for ready access by the processor 1105. In some situations, the electronic storage unit 1115 can be precluded, and machine-executable instructions are stored on memory 1110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 can include or be in communication with an electronic display 1135 that comprises a user interface (UI) 1140 for providing, for example, a portal for viewing one or more videos of a surgical procedure. In some cases, the user interface may be configured to permit one or more end users to view different subsets of the plurality of videos captured by the plurality of imaging devices. The portal may be provided through an application programming interface (API). A user or entity can also interact with various elements in the portal via the UI. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1105. The algorithm may be configured to (a) obtain a plurality of videos of a surgical procedure; (b) determine an amount of progress for the surgical procedure based at least in part on the plurality of videos; and (c) update an estimated timing of one or more steps of the surgical procedure based at least in part on the amount of progress. The algorithm may be further configured to provide the estimating timing to one or more end users to coordinate another surgical procedure or patient room turnover. In some cases, the algorithm may be configured to (a) obtain a plurality of videos of a surgical procedure, wherein the plurality of videos are captured using a plurality of imaging devices; and (b) provide the plurality of videos to a plurality of end users, wherein each end user of the plurality of end users receives a different subset of the plurality of videos.

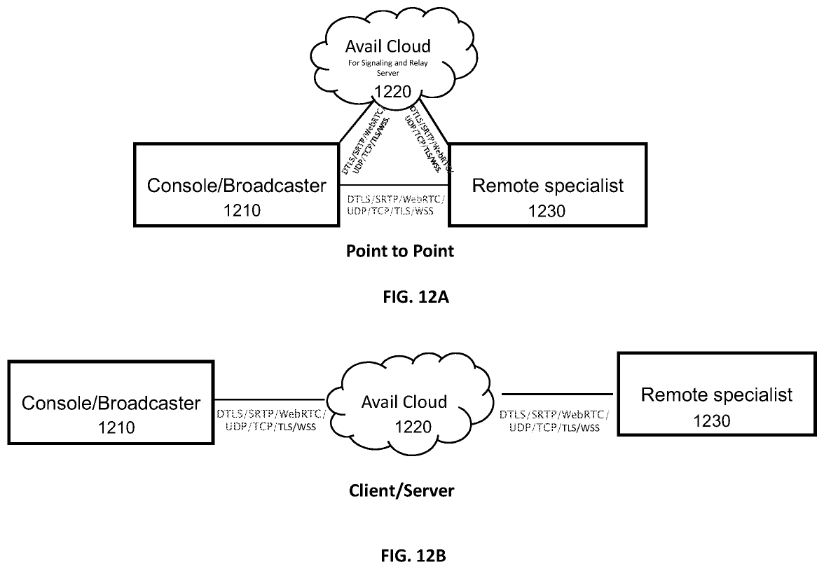

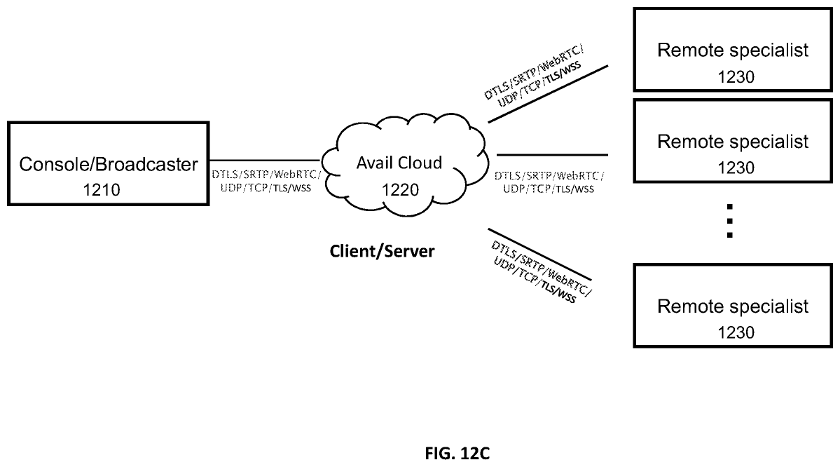

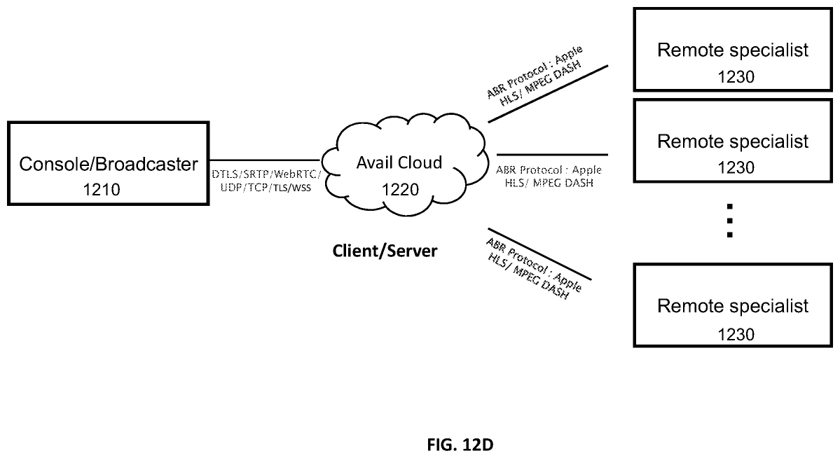

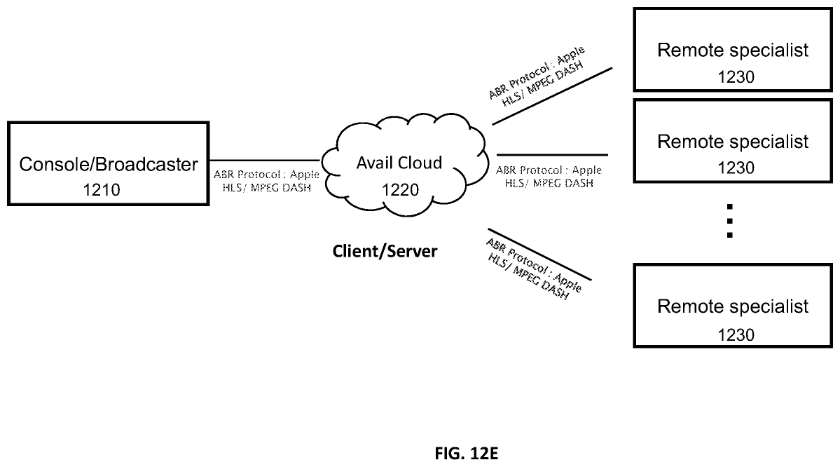

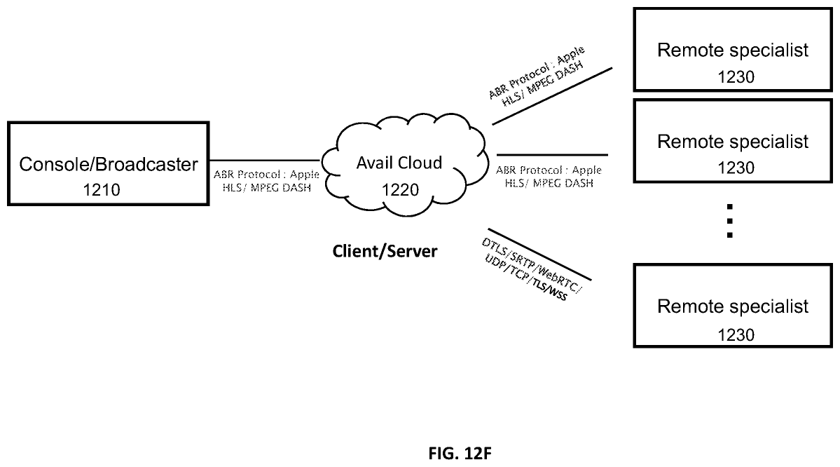

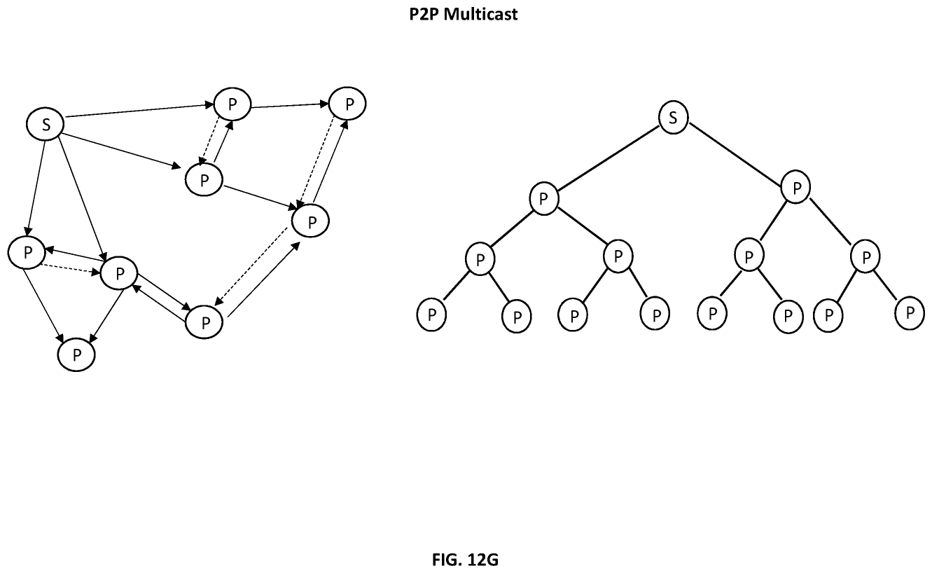

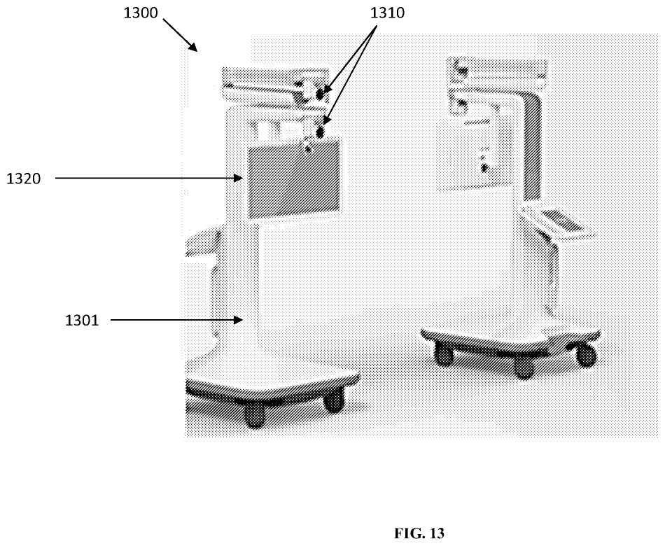

FIGS. 12A, 12B, 12C, 12D, 12E, 12F, and 12G illustrate various non-limiting embodiments for streaming a plurality of videos to one or more end users. Various methods for streaming a plurality of videos to one or more end users may be implemented using a video streaming platform. The video streaming platform may comprise a console or broadcaster 1210 that is configured to stream one or more videos from the console 1210 to one or more end users or remote specialists 1230 using a client/server 1220, peer-to-peer (P2P) computing or networking, P2P multicasting, and/or a combination of client/server streaming and P2P multicasting methods.

FIG. 12A illustrates a method of point to point video streaming that may be used to stream one or more videos from a cloud server 1220 to a console 1210 and/or a remote specialist 1230. The cloud server 1220 may be configured to operate as a signaling and relay server. In some cases, the console 1210 may be configured to stream the one or more videos directly to the remote specialist 1230. The one or more videos may be streamed using one or more streaming protocols and technologies such as Secure Real-Time Transport Protocol (SRTP), Real-Time Transport Protocol (RTP), Real Time Streaming Protocol (RTSP), Datagram Transport Layer Security (DTLS), Session Description Protocol (SDP), Session Initiation Protocol (SIP), Web Real-Time Communication (WebRTC), Transport Layer Security (TLS), WebSocket Secure (WSS), Real-Time Messaging Protocol (RTMP), User Datagram Protocol (UDP), Transmission Control Protocol (TCP), and/or any combination thereof.

FIG. 12B illustrates a method of client/server video streaming that may be used to stream one or more videos to a remote specialist. In some cases, a console 1210 may be configured to stream the one or more videos to a cloud server 1220. The cloud server 1220 may be configured to stream the one or more videos to a remote specialist 1230. As described above, the one or more videos may be streamed using one or more streaming protocols and technologies such as Secure Real-Time Transport Protocol (SRTP), Real-Time Transport Protocol (RTP), Real Time Streaming Protocol (RTSP), Datagram Transport Layer Security (DTLS), Session Description Protocol (SDP), Session Initiation Protocol (SIP), Web Real-Time Communication (WebRTC), Transport Layer Security (TLS), WebSocket Secure (WSS), Real-Time Messaging Protocol (RTMP), User Datagram Protocol (UDP), Transmission Control Protocol (TCP), and/or any combination thereof.

FIG. 12C illustrates an example of a console 1210 that may be configured to capture or receive data and/or videos from one or more medical imaging devices or cameras that are connected or operatively coupled to the console 1210. The console 1210 may be configured to create a single composed frame from the data and/or videos captured by or received from the one or more medical imaging devices or cameras. The single composed frame may be sent from the console 1210 to a plurality of remote participants 1230 via a cloud server 1220. One or more policies for sharing or viewing the videos or video frames may be defined at a broadcast level (e.g., at the console 1210), in the cloud server 1220, or at a remote user level (e.g., at an end user device of a remote participant or specialist 1230). The one or more policies may be used to determine which parts of a video or a video frame is of interest or relevant to each end user or remote specialist 1230. In some cases, the cloud server 1220 may be configured to modify (e.g., crop and/or enhance) the one or more videos or video frames and to send the one or more modified videos or video frames to each remote participant or specialist 1230 based on the one or more policies or rules defining which portions of the videos or video frames broadcasted by the console 1210 may be viewed or accessed by each remote specialist 1230. Based on the one or more policies or rules in place for viewing and accessing the one or more videos or video frames, the broadcaster or console 1210 may be configured to multiplex multiple independent streams that are targeted to different end users or remote specialists 1230 via the cloud server 1220 or directly using peer-to-peer (P2P) networking. In addition, the console 1210 or the cloud server 1220 may be configured to define or select one or more distinct regions of interest (ROI) within the videos or video frames for streaming to different remote users, based on the one or more policies or rules for viewing and accessing the one or more videos or video frames. Such a system may be configured to segment or partition different portions of a video or a video frame and to enable the distribution of the different portions of the videos or video frames to different end users, thereby enhancing security and privacy. The distribution of different portions of the videos or video frames to different end users may also enhance focus and clarity by allowing different end users to easily monitor different aspects or steps of a surgical procedure or track different tools used to perform one or more steps of a surgical procedure. The different portions of the videos or video frames streamed from the console 1210 may be tailored to each end user or remote specialist 1230 depending on a role of each end user or remote specialist 1230 and/or a relevance of the different portions of the videos or video frames to each end user or remote specialist 1230.

As shown in FIG. 12C, the one or more videos or video frames and/or the different segmented portions of the one or more videos or video frames may be broadcasted from the console 1210 to the cloud server 1220 using one or more streaming protocols and technologies such as Secure Real-Time Transport Protocol (SRTP), Real-Time Transport Protocol (RTP), Real Time Streaming Protocol (RTSP), Datagram Transport Layer Security (DTLS), Session Description Protocol (SDP), Session Initiation Protocol (SIP), Web Real-Time Communication (WebRTC), Transport Layer Security (TLS), WebSocket Secure (WSS), Real-Time Messaging Protocol (RTMP), User Datagram Protocol (UDP), Transmission Control Protocol (TCP), and/or any combination thereof. The one or more videos or video frames and/or the different segmented portions of the one or more videos or video frames may be streamed from the cloud server 1220 to a plurality of remote specialists 1230 using one or more streaming protocols and technologies such as Secure Real-Time Transport Protocol (SRTP), Real-Time Transport Protocol (RTP), Real Time Streaming Protocol (RTSP), Datagram Transport Layer Security (DTLS), Session Description Protocol (SDP), Session Initiation Protocol (SIP), Web Real-Time Communication (WebRTC), Transport Layer Security (TLS), WebSocket Secure (WSS), Real-Time Messaging Protocol (RTMP), User Datagram Protocol (UDP), Transmission Control Protocol (TCP), and/or any combination thereof.

As shown in FIG. 12D, the one or more videos or video frames and/or the different segmented portions of the one or more videos or video frames may be broadcasted from the console 1210 to the cloud server 1220 using one or more streaming protocols and technologies such as Secure Real-Time Transport Protocol (SRTP), Real-Time Transport Protocol (RTP), Real Time Streaming Protocol (RTSP), Datagram Transport Layer Security (DTLS), Session Description Protocol (SDP), Session Initiation Protocol (SIP), Web Real-Time Communication (WebRTC), Transport Layer Security (TLS), WebSocket Secure (WSS), Real-Time Messaging Protocol (RTMP), User Datagram Protocol (UDP), Transmission Control Protocol (TCP), and/or any combination thereof. In some cases, the one or more videos or video frames and/or the different segmented portions of the one or more videos or video frames may be broadcasted from the cloud server 1220 to a plurality of remote specialists 1230 using HyperText Transfer Protocol (HTTP) adaptive bitrate streaming (ABR), Apple™ HTTP Live Streaming (HLS), Moving Picture Experts Group Dynamic Adaptive Streaming over HTTP (MPEG-DASH), Microsoft™ Smooth Streaming, Adobe™ HTTP Dynamic Streaming (HDS), Common Media Application Format (CMAF), and/or any combination thereof. Apple™ HLS, MPEG-DASH, and CMAF may be used in combination with chunked transfer encoding to support low latency streaming. As used herein, low latency streaming may refer to streaming of videos or video frames with a latency (i.e., a delay between video capture and video streaming) that is about 10 seconds, 9 seconds, 8 seconds, 7 seconds, 6 seconds, 5 seconds, 4 seconds, 3 seconds, 2 seconds, 1 second, 1 millisecond, 1 microsecond, 1 nanosecond, or less.

As shown in FIG. 12E and FIG. 12F, in some cases, the one or more videos or video frames and/or the different segmented portions of the one or more videos or video frames may be broadcasted from the console 1210 to the cloud server 1220 using HyperText Transfer Protocol (HTTP) adaptive bitrate streaming (ABR), Apple™ HTTP Live Streaming (HLS), Moving Picture Experts Group Dynamic Adaptive Streaming over HTTP (MPEG-DASH), Microsoft™ Smooth Streaming, Adobe™ HTTP Dynamic Streaming (HDS), Common Media Application Format (CMAF), and/or any combination thereof. In some cases, the one or more videos or video frames and/or the different segmented portions of the one or more videos or video frames may be broadcasted from the cloud server 1220 to one or more remote specialists 1230 using HyperText Transfer Protocol (HTTP) adaptive bitrate streaming (ABR), Apple™ HTTP Live Streaming (HLS), Moving Picture Experts Group Dynamic Adaptive Streaming over HTTP (MPEG-DASH), Microsoft™ Smooth Streaming, Adobe™ HTTP Dynamic Streaming (HDS), Common Media Application Format (CMAF), and/or any combination thereof. In other cases, the one or more videos or video frames and/or the different segmented portions of the one or more videos or video frames may be broadcasted from the cloud server 1220 to one or more remote specialists 1230 using Secure Real-Time Transport Protocol (SRTP), Real-Time Transport Protocol (RTP), Real Time Streaming Protocol (RTSP), Datagram Transport Layer Security (DTLS), Session Description Protocol (SDP), Session Initiation Protocol (SIP), Web Real-Time Communication (WebRTC), Transport Layer Security (TLS), WebSocket Secure (WSS), Real-Time Messaging Protocol (RTMP), User Datagram Protocol (UDP), Transmission Control Protocol (TCP), and/or any combination thereof. In any of the embodiments described herein, the one or more videos or video frames and/or the different segmented portions of the one or more videos or video frames may be broadcasted from the cloud server 1220 to different remote specialists 1230 using different streaming protocols.

FIG. 12G illustrates examples of peer-to-peer multicast streaming methods that may be used to stream one or more videos captured by a plurality of imaging devices to a plurality of end users. In some cases, the one or more videos may be streamed from a streaming source (e.g., a console or a broadcaster) to a plurality of peers or end users. In some cases, one or more peers in a network may stream the one or more videos to other peers in the network.

In any of the embodiments described herein, one or more video codecs may be used to stream the one or more videos captured by the plurality of imaging devices. The one or more video codecs may include High Efficiency Video Coding (HEVC or H.265), Advanced Video Coding (AVC or H.264), VP9, or AOMedia Video 1 (AV1). In any of the embodiments described herein, one or more audio may be used to stream audio associated with the one or more videos. The one or more audio codecs may include G.711 PCM (A-law), G.711 PCM (μ-law), Opus, Advanced Audio Coding (AAC), Dolby Digital AC-3, or Dolby Digital Plus (Enhanced AC-3). In any of the embodiments described herein, the videos or video frames captured by the medical imaging devices and cameras connected or operatively coupled to the broadcasting console may be rendered, captured, composed, anonymized, encoded, encrypted, and/or streamed to one or more remote participants using any of the protocols and codecs described herein.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

We claim:

1. A method for video collaboration, the method comprising:
(a) obtaining a plurality of videos of a surgical procedure;
(b) determining an amount of progress for one or more steps of the surgical procedure based at least in part on the plurality of videos or a subset thereof; and
(c) updating an estimated timing for performing or completing the one or more steps of the surgical procedure based at least in part on the amount of progress determined in step (b).

2. The method of claim 1, further comprising providing the estimated timing to one or more end users to coordinate a performance or a completion of the surgical procedure or at least one other surgical procedure that is different than the surgical procedure.

3. The method of claim 1, further comprising providing the estimated timing to one or more end users to coordinate patient room turnover, and scheduling or updating a scheduling for one or more other surgical procedure based on the estimated timing for performing or completing the one or more steps of the surgical procedure.

4. The method of claim 2, wherein the surgical procedure and the at least one other surgical procedure comprise two or more medical operations involving a donor subject and a recipient subject.

5. (canceled)

6. The method of claim 3, wherein scheduling the one or more other surgical procedures comprises identifying or assigning an available time slot or an available operating room for the one or more other surgical procedures.

7. The method of claim 1, wherein determining the amount of progress for the one or more steps of the surgical procedure comprises analyzing the plurality of videos to track a movement or a usage of one or more tools used to perform the one or more steps of the surgical procedure.

8. (canceled)

9. The method of claim 1, further comprising generating a visual status bar based on the updated estimated timing, wherein the visual status bar indicates a total predicted time to complete the one or more steps of the surgical procedure.

10. The method of claim 1, further comprising generating an alert or a notification when the estimated timing deviates from a predicted timing by a threshold value.

11. (canceled)

12. The method of claim 10, wherein the threshold value is adjustable based on a type of procedure or a level of experience of an operator performing the surgical procedure.

13. (canceled)

14. The method of claim 1, further comprising determining an efficiency of an operator performing the surgical procedure based at least in part on an updated estimated timing to complete the one or more steps of the surgical procedure.

15.-16. (canceled)

17. A method for video collaboration, the method comprising:
(a) obtaining a plurality of videos of a surgical procedure, wherein the plurality of videos are captured using a plurality of imaging devices; and
(b) providing the plurality of videos to a plurality of end users, wherein at least one end user of the plurality of end users receives a different portion or subset of the plurality of videos than at least one other end user of the plurality of end users, based on an identity, an expertise, or an availability of the at least one end user.

18. (canceled)

19. The method of claim 17, wherein providing the plurality of videos comprises streaming or broadcasting the plurality of videos to the plurality of end users in real time as the plurality of videos are being captured by the plurality of imaging devices.

20.-22. (canceled)

23. The method of claim 17, wherein a first video is captured using a first imaging device of the plurality of imaging devices and wherein a second video is captured using a second imaging device of the plurality of imaging devices.

24. The method of claim 23, wherein the second imaging device provides a different view of the surgical procedure than the first imaging device.

25.-30. (canceled)

31. The method of claim 17, wherein the plurality of videos are stored or compiled in a video library, wherein providing the plurality of videos comprises broadcasting, streaming, or providing access to one or more of the plurality of videos through one or more video on demand services or models.

32. The method of claim 17, further comprising implementing a virtual session for the plurality of end users to collaboratively view and provide one or more annotations for the plurality of videos in real time as the plurality of videos are being captured.

33.-36. (canceled)

37. A method for video collaboration, the method comprising:
(a) providing one or more videos of a surgical procedure to a plurality of users; and
(b) providing a virtual workspace for the plurality of users to collaborate based on the one or more videos, wherein the virtual workspace permits each of the plurality of users to (i) view the one or more videos or capture one or more recordings of the one or more videos, (ii) provide one or more telestrations to the one or videos or recordings, and (iii) distribute the one or more videos or recordings comprising the one or more telestrations to the plurality of users.

38. The method of claim 37, wherein the virtual workspace permits the plurality of users to simultaneously stream the one or more videos and distribute the one or more videos or recordings comprising the one or more telestrations to the plurality of users.

39. The method of claim 38, wherein the virtual workspace permits a first user to provide a first set of telestrations and a second user to provide a second set of telestrations simultaneously.

40. The method of claim 39, wherein the virtual workspace permits a third user to simultaneously view the first set of telestrations and the second set of telestrations to compare or contrast inputs or guidance provided by the first user and the second user.

41.-48. (canceled)
